(19) United States
(12) Patent Application Publication
(10) Pub. No.: US 2024/0060960 A1
(43) Pub. Date: Feb. 22, 2024

(54) METHODS OF PREDICTING PROGRESSION OF NEUROLOGICAL DISEASE

(71) Applicants: RESEARCH INSTITUTE AT NATIONWIDE CHILDREN'S HOSPITAL, Columbus, OH (US); THE UNIVERSITY OF SHEFFIELD, Sheffield (GB)

(72) Inventors: Kathrin Christine Meyer, Columbus, OH (US); Cassandra Nicole Dennys-Rivers, Westerville, OH (US); Laura Ferraiuolo, Sheffield (GB)

(73) Assignees: RESEARCH INSTITUTE AT NATIONWIDE CHILDREN'S HOSPITAL, Columbus, OH (US); THE UNIVERSITY OF SHEFFIELD, Sheffield (GB)

(21) Appl. No.: 18/271,980

(22) PCT Filed: Jan. 13, 2022
(86) PCT No.: PCT/US22/12221

§ 371 Date: Jul. 12, 2023

Related U.S. Application Data
(60) Provisional application No. 63/137,077, filed on Jan. 13, 2021.

(51) Int. Cl. G01N 33/50

(52) U.S. Cl. CPC G01N 33/5005; G01N 2800/56; G01N 2800/52

(57) ABSTRACT

The disclosure provides for methods of predicting disease progression comprising detecting a modification of one or more disease markers in a glial cell or neuronal cell generated from a skin cell of the subject. The disclosed methods include methods of identifying subjects that are responsive to a therapeutic agent and methods of determining effectiveness of a therapeutic agent.

This application claims priority benefit to U.S. Provisional Application No. 63/137,077, filed Jan. 13, 2021, which is incorporated by reference herein in its entirety.

### FIELD

The disclosure provides for methods of predicting disease progression comprising detecting a modification of one or more disease markers in a glial cell or neuronal cell generated from a skin cell of the subject. The disclosed methods include methods of identifying subjects that are responsive to a therapeutic agent and methods of determining effectiveness of a therapeutic agent.

### BACKGROUND

The generation of new models to study neurological and neurodegenerative diseases is a major challenge. Each disorder may be caused by a variety of mutation and the progression and/or severity of the disease may vary depending on the mutation. Thus, predictable models are rare.

For example, in Amyotrophic Lateral Sclerosis (ALS) is one such neurodegenerative diseases. 90% of the cases are sporadic without familial inheritance pattern (sALS) and less than 20% of all cases can be linked to mutations in more than 20 different genes (fALS). This variability of cause and the diverse pattern of disease courses with different onset points and progression rates might in part be responsible for the low success of clinical trials. The development of new therapeutics as well as disease mechanistic studies might improve drastically if patients could be sub-grouped based on different disease characteristics. Compelling evidence has demonstrated that non-neuronal cells are highly involved in the mechanisms leading to motor neuron (MN) death in ALS. Astrocytes were shown to be implicated in setting the pace for disease progression, while the role of oligodendrocytes remains less clear. Recent studies demonstrated the presence of misfolded super oxide dismutase 1 (SOD1) in ALS patient post-mortem biopsies. While SOD1 is clearly a target in cases carrying mutations in this gene, it is still debated whether sALS patients and patients carrying other mutations could benefit from the same treatment.

Thus, the heterogeneous nature of neurodegenerative disorders, such as ALS, is not effectively represented in a single transgenic mouse model and utilizing such model systems risks developing a targeted therapy for a small, mutation specific, patient subpopulation. Most clinical trials do not account for these patient subpopulations in trial design which makes interpretation of patient outcomes extremely difficult. This is one reason why many effective preclinical treatments for ALS fail in clinical trial.

There is a need for methods of analyzing the progression of a neurological and neurodegenerative disease and methods of predicting responsiveness to therapeutic agents in patients suffering neurological disease or neurodegenerative disorder. There is also a need for stratifying subpopulations of patients suffering neurodegenerative disease in order to improve clinical trials.

### SUMMARY

Currently, there is no human in vitro modeling system for neurological and neurodegenerative diseases that can be used as a predictor of disease prognosis, to subgroup patients, or to select patients for clinical trials or to identify best treatment options for them. Disclosed herein are methods that use a direct conversion assay to fill this gap in the medical field.

The disclosed methods utilize a fast and efficient reprogramming method that allows for generating induced neuronal progenitor cells (iNPCs) from patient skin derived fibroblasts. These cells readily differentiate into astrocytes and oligodendrocytes and they display previously reported disease phenotypes and decrease motor neuron survival in co-culture to variable extents. Therefore, these cells represent a promising tool for studying disease mechanisms and potential therapeutics in ALS.

Patient diversity and unknown disease cause is a major challenge for drug development and clinical trial design. For example, heterogeneity in ALS patients (sALS and fALS) is not reflective in current animal models used to evaluate therapies and therefore, the direct translation of potential therapeutics using these models have proven difficult. Direct reprogramming facilitates compound screening on sALS and fALS lines and therefore the data presented herein indicate diverse patient response to therapeutic agent suggesting shared pathways between patient subgroups. The cell lines disclosed herein may be used to identify patient responders to therapies and distinguish them from nonresponders. An evaluation of ALS disease markers identified increased mitochondrial activity as the shared parameter unique to responders.

Patient iAstrocytes can be used to identify both disease modifiers and pathways dysregulated in a given individual potentially predicting therapeutic responsiveness, e.g., elevation in mitochondrial activity (basal and ATP linked respiration) may indicate if a patient is a strong candidate for therapies. An enhanced understanding of cellular profiles could aid clinicians in determining best treatment approach for patients. The role of astrocytes in providing metabolic support for neurons and regulating neurotransmission suggests that these cells, in addition to neurons, may be a potential therapeutic target in patients with neurological disorders.

The disclosure provides for methods of predicting progression of a neurological or neurodegenerative disease in a subject in need comprising detecting a modification of one or more disease markers in a glial cell or neuronal cell generated from a skin cell of the subject, compared to a glial cell or neuronal generated from a control cell.

In another embodiment, the disclosure provides for methods of identifying a subject who is responsive to a therapeutic agent, comprising detecting a modification of one or more disease markers in a glial cell or neuronal cell generated from a skin cell of the subject, compared to a glial cell or neuronal cell generated from a control cell and wherein the modification is indicative of cells that are responsive to the therapeutic agent.

In a further embodiment, the disclosure provides for methods of determining effectiveness of a therapeutic agent in a subject, comprising detecting a modification of one or more disease markers in a glial cell or neuronal cell generated from a skin cell of the subject obtained from the subject after administration of the therapeutic agent, compared to a modification of one or more disease markers in a glial cell or neuronal cell generated from a skin cell of the subject obtained from the subject before administration of the therapeutic agent.

In any of the disclosed methods, the glial cell or neuronal cell is differentiated from a neuron progenitor cell derived from a skin cell obtained from the subject. For example, the glial cell is an astrocyte, microglia or oligodendrocyte. In addition, in any of the disclosed methods, the skin cell is a fibroblast.

In any of the disclosed methods, the modification may be an increase in one or more disease markers, and wherein the increase is indicative of an increased progression. Alternatively, in any of the methods, the modification is a decrease in one or more disease markers disease markers, wherein the decrease is indicative of an increased progression.

The term “modification” refers to a change in the level or amount of a disease marker or a change in the expression of a disease marker. This change is determined by a comparison to a level or amount of the disease marker in a control cell. In some aspects, the modification is an increase or decrease of the expression, level or amount of the disease marker compared to a control cell. An increase may be due to an induction of the expression of a disease marker that is generally not expressed in a cell from a healthy subject. A decrease may be a reduction in the level of expression of the disease marker or a reduction in the level or amount of disease marker with or without a change in expression. The term “decrease” refers to a complete elimination or abrogation of the expression or amount of the marker, or a reduction in the rate of expression of the marker without a complete abrogation of expression of the marker. The term “disease decrease” also refers to a reduction in the amount or level of the disease marker without a complete abrogation of the disease marker.

In exemplary embodiment, the disease marker of any of the disclosed methods is a marker for Alzheimer's disease, Parkinson's disease, Multiple Sclerosis, Amyotrophic Lateral Sclerosis (ALS), other demyelination related disorders, senile dementia, subcortical dementia, arteriosclerotic dementia, AIDS-associated dementia, or other dementias, a central nervous system cancer, traumatic brain injury, spinal cord injury, stroke or cerebral ischemia, cerebral vasculitis, epilepsy, Huntington's disease, Rett Syndrome, Pitt Hopkins Syndrome, SMARD1/CMT25, Tourette's syndrome, Guillain Barre syndrome, Wilson disease, Pick's disease, neuroinflammatory disorders, SCN2A-related disorders, encephalitis, encephalomyelitis or meningitis of viral, fungal or bacterial origin, or other central nervous system infections, prion diseases, cerebellar ataxias, cerebellar degeneration, spinocerebellar degeneration syndromes, Friederichs ataxia, ataxia telangiectasia, spinal dystrophy, spinal muscular atrophy, NEDAMSS, SLC6A1-related disorders, SCN1A-related disorder, IRF2BPL-related disorders, progressive supranuclear palsy, dystonia, muscle spasticity, tremor, retinitis pigmentosa, striatonigral degeneration, mitochondrial encephalo-myopathies, neuronal ceroid lipofuscinosis, Batten Disease, hepatic encephalopathies, renal encephalopathies, metabolic encephalopathies, toxin-induced encephalopathies, or radiation-induced brain damage.

In some embodiments, the disease marker is abnormal mitochondria morphology, change in basal respiration, change in ATP linked respiration, glutamate, or in vitro motor neuron toxicity.

In an exemplary embodiment, the disease marker is a marker of ALS, and the marker is p62, SOD1, misfolded SOD1, BIP, TDP43, abnormal mitochondria morphology, change in basal respiration, change in ATP linked respiration, mi-RNA-146, glutamate, or in vitro motor neuron toxicity.

In another exemplary embodiment, the disease marker is a marker of Batten Disease and the marker is accumulation of auto fluorescent storage material, abnormal mitochondria morphology, change in basal respiration, change in ATP linked respiration, or in vitro motor neuron toxicity.

In a further exemplary embodiment, the disease marker is a marker of a SCN2A-related disorder and the marker is nitric oxide or superoxide, abnormal mitochondria morphology, change in basal respiration, change in ATP linked respiration, or in vitro motor neuron toxicity.

In another embodiment, the disease marker is a marker of NEDAMSS and the marker is wnt1, IRF2BPL, abnormal mitochondria morphology, change in basal respiration, change in ATP linked respiration, or in vitro motor neuron toxicity.

In another embodiment, the disease marker is a marker of SLC6A1-related disorder and the marker is GABA uptake and release, abnormal mitochondria morphology, change in basal respiration, change in ATP linked respiration, or in vitro motor neuron toxicity.

In another embodiment, the disease marker is a marker of SCN1A-related disorder and the marker abnormal mitochondria morphology, change in basal respiration, change in ATP linked respiration, or in vitro motor neuron toxicity.

In an exemplary embodiment, the disease marker is a marker of Rett Syndrome and a marker is set out in Table 5-21 or in vitro motor neuron toxicity.

In another exemplary embodiment, the disease marker is a marker of Pitt Hopkins Syndrome and is a marker TCF-4 or in vitro motor neuron toxicity.

In some embodiments, any of the disclosed methods further comprise the step of obtaining skin cells from the subject. In addition, any of the disclosed methods further comprise the step of generating induced neuronal progenitor cells (iNPCs) from skin cells obtained from the subject. In a further embodiment, any of the disclosed methods further comprises the step of differentiating iNPCs to iAstrocytes and/or neurons and/or oligodendrocytes.

In any of the disclosed methods, the subject is suffering from, is at risk of suffering from or has been diagnosed with Alzheimer's disease, Parkinson's disease, Multiple Sclerosis, Amyotrophic Lateral Sclerosis (ALS), other demyelination related disorders, senile dementia, subcortical dementia, arteriosclerotic dementia, AIDS-associated dementia, or other dementias, a central nervous system cancer, traumatic brain injury, spinal cord injury, stroke or cerebral ischemia, cerebral vasculitis, epilepsy, Huntington's disease, Rett Syndrome, Pitt Hopkins Syndrome, SMARD1/CMT25, Tourette's syndrome, Guillain Barre syndrome, Wilson disease, Pick's disease, neuroinflammatory disorders, SCN2A-related disorders, encephalitis, encephalomyelitis or meningitis of viral, fungal or bacterial origin, or other central nervous system infections, prion diseases, cerebellar ataxias, cerebellar degeneration, spinocerebellar degeneration syndromes, Friedreichs ataxia, ataxia telangiectasia, spinal dysmyotrophy, spinal muscle atrophy, NEDAMSS, progressive supranuclear palsy, dystonia, muscle spasticity, tremor, retinitis pigmentosa, striatonigral degeneration, mitochondrial encephalo-myopathies, neuronal ceroid lipofuscinosis such as Batten Disease, hepatic encephalopathies, renal encephalopathies, metabolic encephalopathies, toxin-induced encephalopathies, or radiation-induced brain damage.

In an exemplary embodiment, in any of the disclosed methods, the subject has been diagnosed with ALS or has a mutation in SOD1, TDP43, C90RF72 or FUS.

In another exemplary embodiment, in any of the disclosed methods, the subject has been diagnosed with Batten Disease or neuronal ceroid lipofuscinosis, or has a mutation in CLN1 gene, CLN2 gene, CNL3 gene, CLN4 gene, CLN5 gene, CLN6 gene, CLN7, CLN8 or CLN10.

In another exemplary embodiment, in any of the disclosed methods, the subject has a SCN2A mutation, a mutated SCN2A voltage-gated sodium channel protein a SLC6A1 mutation, a SCN1A mutation or a mutation in IRF2BPL.

### BRIEF DESCRIPTION OF THE DRAWINGS

FIG. 1A-1C provides a schematic of the conversion of fibroblasts to induced astrocytes (iAstrocytes). A). An overlook of the direct differentiation process B). Skin fibroblasts are converted to neuronal progenitor cells (Meyer et al, PNAS, 2014), and then differentiated into iAstrocytes. C). The differentiated cells are then used for co-culture and metabolic analysis experiments.

FIG. 2A-2B demonstrate that iAstrocytes from different ALS subpopulations vary in toxicity to motor neurons.

FIG. 3 provides representative images of ALS patient iAstrocytes display stained for ALS disease markers: p62, Mitotracker Red, NO₂ and BIP.

FIG. 4 demonstrates that elevation of p62 and formation of aggregated did not correlate with iAstrocyte severity in ALS patient iAstrocytes.

FIG. 5 demonstrates that ALS patient iAstrocytes had abnormal mitochondrial morphology. Representative images (60×) using MitoTracker Red staining indicate abnormal mitochondrial morphology in ALS patient lines

FIG. 6A-6C demonstrates that ALS patient iAstrocytes have a differential level of basal (A), and ATP-linked respiration (B) and complex IV (COX IV) activity (C). Statistical analysis performed using One-way ANOVA (N=3 by quintuplicate) compared against internal standard line (Ctl1).

FIG. 7 provides quantification of BIP staining in different subpopulations of ALS iAstrocytes. The expression of BIP correlates with severity of ALS patient cell lines.

FIG. 8 provides a list of the ALS patient cell lines and a graph indicating the toxicity index of the iAstrocytes on motor neurons.

FIG. 9A-9C are graphs showing cellular glycolysis and mitochondrial coupling is not significantly different between most patient lines. A.) iAstrocytes were seeded on a 24 or 96 well Seahorse plate for extracellular flux analysis. Oxygen consumption rate (OCR) and extracellular acidification rate (ECAR) were used to calculate glycolytic protein efflux rate (glycoPER), which quantifies media acidification by lactate production. Representative ECAR rate graph. B.) Total cellular glycolysis is measured following mitochondrial shutdown (difference between basal and AA/Rot injection). C.) Percent mitochondrial coupling was calculated using values from basal and/or ATP-linked respiration. The data indicates that total cellular glycolysis and percent coupled mitochondria may be a potential parameter for patient stratification and drug response. Dotted line represents average control values. Data represents a minimum of 3 independent experiments. Statistical analysis was performed using one-way ANOVA comparing average controls.

FIG. 10A-10F show the metabolism of ALS patient iAstrocytes is highly distinct. iAstrocytes were seeded on a 96 well Seahorse plate for extracellular flux analysis. Rate graphs for mitochondrial dependency on glycolysis (FIG. 10A) fatty acid oxidation (FIG. 10B) and glutaminolysis (FIG. 9C) is shown. Mitochondrial dependency is calculated by measuring basal OCR for three time points followed by injections of pathway specific inhibitors (UK5099, etomoxir, BPTES). The difference in OCR following inhibitor addition determines mitochondrial fuel dependency on specific pathway tested. In this case, mitochondrial dependency on glycolysis (FIG. 10D), fatty acid oxidation (FIG. 10E) and glutamine (FIG. 10F) is calculated. The combined mitochondrial metabolic dependency profile is patient line specific Dotted line represents average control values. Data represents a minimum of 3 independent experiments. Statistical analysis was performed using one-way ANOVA comparing to the average controls (FIGS. 10D, 10E and 10F).

FIG. 11A-11C demonstrates increased total SOD1 levels and misfolded SOD1 levels in ALS iAstrocytes from patients with milder disease progression. A) Representative Immunofluorescent Western Blot showing misfolded SOD1 identified by the B8H10 antibody (red, upper lane), total SOD1 in green and actin in red lower lane. B) Quantification and summary of several western blots indicating that less severe ALS patient cell lines contain higher levels of total SOD1. C) The same cell lines as in B were also analyzed for levels of misfolded SOD1 detected by B8H10 antibody and put in the same order as in B. The data indicates higher levels of misfolded SOD1 in milder ALS cases compared to more severe patient cell lines. Disease Controls also showed elevated misfolded SOD1 levels.

FIG. 12A-12B provides native page Western blot indicates higher levels of misfolded SOD1 in several cell lines from ALS patients. A.) Representative native page immunoblots showing misfolded SOD1 identified by the B8H10 antibody (green, upper lane). Lower red square shows a representative area of the whole western blot that was used for quantifying total protein levels. For each sample, the whole lane was used for quantification of total protein. B.) Quantification and summary of native page immunoblots showing elevated levels of misfolded SOD1 in tested cell lines from ALS patients. Of note, the cell lines we selected here have a severe impact on motor neuron survival in vitro and did not show an elevation in misfolded SOD1 levels in the denaturing western blot conditions. This could be due to the type of normalization, the milder lysis protocol or the running buffer conditions, as protein analysis can vary depending on the chosen parameters.

FIG. 13A-13B demonstrate iAstrocytes derived from fALS and sALS patient-fibroblasts decrease the viability of HB9/GFP-positive motor neurons. A.) Representative images of HB9/GFP expressing MNs (shown in black) after 96 h in co-culture with iAstrocytes. A notable decrease in MN viability was observed when exposed to iAstrocytes derived from both sALS and fALS-patients. B) Relative percentage of HB9/GFP-positive MN survival after 96 h in co-culture with iAstrocytes from ALS patients compared to iAstrocytes from non-ALS controls (CTR1 and CTR2). Results are mean values ±SEM cell survival rate from at least five independent experiments. **p<0.01 vs. average controls, one-way ANOVA followed by Dunnett's post hoc test; fALS, familial amyotrophic lateral sclerosis (ALS); sALS, sporadic ALS; SOD1, superoxide dismutase 1. Scale bar represents 200 μm.

FIG. 14A-14C demonstrate decreased GFAP expression was observed in iAstrocytes from 2 fALS patients together with increased Cx43 in female ALS individuals and elevated cell proliferation in all less 1 fALS case. A) Expression of glial fibrillary acidic protein. B) connexin-43 (Cx43) assessed by Western blot analysis in iAstrocytes. Representative results from one blot are shown. 13-actin was used as loading control. C) Proliferation of iAstrocytes was determined by Ki-67 staining and the number of Ki-67 (red) positive cells counted using the IN CELL developer and analyzer software. Cell cytoplasm was stained with CD44 astrocyte marker (green). DAPI (blue) was used for nuclei staining. Results are mean values ±SEM fold change vs. non-ALS controls from at least five independent experiments. *p<0.05, **p<0.01 and ***p <0.001 vs. non-ALS controls (CTR1 and CTR2), one-way ANOVA followed by Dunnett's post hoc test; fALS, familial amyotrophic lateral sclerosis; sALS, sporadic ALS; SOD1, superoxide dismutase 1. Scale bar represents 50 μm.

FIG. 15A-15D demonstrate iAstrocytes derived from fALS and sALS patient-fibroblasts show deregulated miRNAs levels. Expression of microRNA (miR)-181b (A), miR-21 (B), miR-155 (C) and miR-146a (D) in iAstrocytes by qRT-PCR. Results are mean values ±SEM fold change vs. non-ALS controls from at least three independent experiments. *p<0.05, **p<0.01 and ***p<0.001 vs. non-ALS controls (CTR1 and CTR2), one-way ANOVA followed by Dunnett's post hoc test; fALS, familial amyotrophic lateral sclerosis; sALS, sporadic ALS; SOD1, superoxide dismutase 1.

FIG. 16A-16D demonstrate aberrant representation of miR-21, miR-155 and miR-146a characterize iAstrocyte-derived small EVs from ALS patients. A) Concentration (particles/mL) and size distribution data of iAstrocyte released small EVs were measured by Nanoparticle Tracking Analysis using NanoSight. Inflammatory-related microRNA (miR)-21 (B), miR-155 (C) and miR-146a (D) in small EVs released by both fALS (ALS2 and ALS3) and sALS (ALS7) iAstrocytes were assessed by qRT-PCR. Results are mean values ±SEM fold change from at least four independent experiments. *p<0.05, **p<0.01 and ***p<0.001 vs. non-ALS controls (CTR1 and CTR2), one-way ANOVA followed by Dunnett's post hoc test. ALS, amyotrophic lateral sclerosis.

FIG. 17A-17C demonstrates SCN2A patient iAstrocyte cells show stable or increased expression of SCN2A and increased sodium and calcium signaling. A) SCN2A patient iAstrocyte cells display increased or stable expression of SCN2A. Quantification of SCN2A expression was done by qPCR. Data shows the fold change difference of expression normalized to the average of controls and is representative of three independent experiments. Data points in scatter plot bar graph represent mean±SEM. B) SCN2A loss of function mutations lead to stable or increased sodium ion influx into the cells (B) as well as increase calcium signaling (C). The integrated density of the fluorescent signal was normalized to the average of controls. Data points in scatter plot with bar graph represent mean±SEM. Statistical analysis was performed using ordinary one-way ANOVA, Dunnett's multiple comparison test (****=P<0.0001, ***=P 0.0001), and is representative of at least three independent experiments.

FIG. 18A-18D demonstrates loss of function SCN2A iAstrocyte cells produce higher levels of nitric oxide and superoxide species. Representative images of iAstrocyte cells showing various levels of nitric oxide (A) and superoxide (C) signal intensity. In cell analyzer was used for imaging cells. Quantification of average fluorescent intensity reading for nitric oxide (B) and superoxide signal (D). Background fluorescent intensity was subtracted from cell fluorescent intensity. These values were then normalized to the average fluorescence intensity of controls. Statistical analysis was performed using ordinary one-way ANOVA and Dunnett's multiple comparison test was performed on all conditions to determine statistical significance (****=P<0.0001). Data points in graph represent mean±SEM and is representative of at least three independent experiments.

FIG. 19A-19B demonstrate that SCN2A iAstrocytes have elevated basal and ATP-linked respiration. iAstrocytes were seeded on a 96 well Seahorse plate for extracellular flux analysis. A) Basal oxygen consumption was measured at three time points followed by ATP synthase inhibition using oligomycin. B) Shows the difference between basal respiration and oligomycin addition was used to calculate ATP linked respiration. All experiments were run at least in triplicate. Statistical analysis was run comparing untreated individual patient lines to control line using one way-ANOVA.

FIG. 20A-20C demonstrate SCN2A patient iNeurons show reduced neurites. A) SCN2A skin fibroblast cells were directly converted to neurons using small chemical compounds in 7 days. Representative brightfield images on day 7 of differentiation show that SCN2A iNeurons have a lower percentage of neuronal survival compared to healthy controls. B) Quantification of neuron survival percentage after 7 days of differentiation. Percent of neurons alive was determined by the amount of neuronal marker Tuj1+ cells remaining at day 7 compared to control. Three to five images were taken per condition and counting was performed by hand. Data was normalized to the average of control and is representative of three independent experiments. Data points in scatter plot with bar graph represent mean±SEM. Statistical analysis was performed using ordinary one-way ANOVA, Dunnett's multiple comparison test C) Representative image of WT GFP-neurons following 3 days in co-culture with patient SCN2A iAstrocyte cells. Scale bar=100 μm.

FIG. 21A-21B demonstrate SCN2A patient iNeurons demonstrate distinct neuronal features. A) Representative brightfield images on day 7 of differentiation show that SCN2A-4 iNeurons. Initial data indicates that neurites/branching varies between these 2 cell lines. B) Representative immunofluorescence staining showing a higher expression of Nav1.2 (green) in SCN2A-4 iNeurons compared to SCN2A-5 iNeurons.

FIG. 22A-22D demonstrate SCN2A expression in brain organoids of patient cell line SCN2A-1 compared to healthy controls. A) Scheme showing the conversion of SCN2A hPSC cells into brain organoids. B) Cells were sorted and clustered according to their expression profiles with merged clusters representing the same broad cell types. Uniform manifold approximation and projection (UMAP) for dimension reduction was used for visualization. C) SCN2A-1 DMSO treated organoids show higher expression of SCN2A (indicated in red) when compared to Controls, particularly within the cortical and the inhibitory interneuron cell clusters; D) Representative immunofluorescence staining showing a higher Nav1.2 expression (green) in SCN2A-1 iNeurons (SCN2A-1 DMSO) compared to healthy control.

FIG. 23A-23C demonstrate auto fluorescent storage material accumulation in fibroblasts from healthy patients (A) and CLN3 patients (B,C).

FIG. 24A-24B provide images demonstrating that CLN3 patient iAstrocytes show abnormal mitochondrial morphology. COX IV (Red) and DAPI(Blue) staining on fibroblasts (FIG. 24A) and iAstrocytes (FIG. 24B) of CLN3 patients.

FIG. 25A-25C demonstrate that there was no difference in the ER stress marker BIP between control and CLN3 iAstrocytes. A.) Immunofluorescence and B.) Western blot analysis of ER N=3

FIG. 26 demonstrates cell line specific changes in mitochondrial activity in CLN3 patient iAstrocytes by A.) basal respiration and B.) ATP linked respiration.

FIG. 27A-27B demonstrate that CLN3 patient istrocytes are toxic to neurons. A) Percent of neuron survival after 3 days of co-culture. B) Representative images of the GFP+ Neurons 3 days after the culture.

FIG. 28A-28B provides data demonstrating the expression of Interferon regulatory factor 2 binding protein like (IRF2BPL) in fibroblasts isolated from healthy individuals and patients suffering from NEDAMSS (denoted as “disease”). A) IRF2BPL protein expression detected by immunofluorescence. B) IRF2BPL protein expression detected by Western blot (n=3). The data indicates no major differences in protein expression levels between healthy controls and NEDAMSS patients except for one patient (P3) which shows a reduction.

FIGS. 29A-29B are representative images showing that IRF2BPL aberrantly accumulates in the cytoplasm of iAST from patients suffering from NEDAMSS (P1. P2, P3 and P4). The blue stain is DAPI and the red staining is IRF2BPL, when overlapping, the stain appears purple.

FIG. 30 provides a graph showing the normalized ratio of number of cells with cytoplasm accumulation of IRF2BPL in fibroblasts to the DAPI counts (n=3).

FIG. 31A-31B provide graphs showing IRF2BPL protein expression in nucleus (FIG. 31A) and cytoplasm (FIG. 31B) extracts from patients and control iAstrocyte (n=3). Quantification supports the observed increase in accumulation in the cytoplasm in patient iASTs and a decrease in nuclear localization seen in immunostaining.

FIG. 32A-32B provides representative images and quantification showing mouse motor neuron survival after three days co-culture of patient or healthy iASTs with GFP+ motor neurons (in black), n=4.

FIG. 33 provides immunofluorescence staining for WNT1 and DAPI in NEDAMSS patient cell lines. WNT1 expression was increased in the NEDAMSS patients.

FIG. 34 provides Western blot data measuring WNT1 levels in the lysate and the supernatant in ASTs from healthy and NEDAMSS patients (n=1). The graph demonstrates that the WNT1 expression is increased in the iAstrocyte supernatants from NEDAMSS patients.

FIG. 35A-35B provide representative images showing that reduced numbers of neurons were found in NEDAMSS patient cell lines compared to healthy controls after direct reprogramming from fibroblasts. Staining using the pan-neuronal marker Tuj1, and FIG. 22B provides staining for neuron specific marker GABA. Control refers to neurons induced from a healthy individual.

FIG. 36 provides graphs showing the comparison of percentage neuronal conversion rate and the neurite length. Indicating reduced generation of neurons from fibroblasts of NEDAMSS patients.

FIG. 37A-37B demonstrate that PTHS iAstrocytes with TCF4 deletions have issues with differentiation. Representative images of iAstrocytes from healthy and TCF4 mutant cells following differentiation are provided (A) in addition to immunostaing data showing reduced staining of Astrocyte marker (GFAP, red) and high level of neural progenitor marker (Nestin, green) in deletion mutation (B)..

FIG. 38A-38B demonstrate that PTHS iAstrocytes with missense mutations have dysregulated TCF4 protein levels whereas deletion mutations have reduced TCF4 levels. Representative Western blots of TCF4 levels within neuronal progenitor cells (A, NPCs) and iAstrocytes (B) show variable expression when normalized against control levels. TCF4 and GAPDH protein levels were quantified and normalized to healthy controls. Importantly, individual with gene deletions show reduction of TCF4 levels. Statistical analysis performed using one-way ANOVA against combined control data (N=3).

FIG. 39A-39B demonstrate PTHS iAstrocytes produce abnormal neurite morphology and decreased motor neuron survival. Representative image of neurons (black) seeded on top of iAstrocytes (A). Neuronal quantification shows reduced survival, skeleton length and average neurite length (B).

### DETAILED DESCRIPTION

The disclosure provides for the use of reprogrammed cells from a patient to identify markers of progression of a neurological disorder. The disclosure provides methods of direct reprogramming of patient skin cells (fibroblasts) to model rare neurodegenerative diseases in vitro. For example, the patient fibroblasts are directly converted into neuronal progenitor cells (iNPCs) then further differentiate these cells into iAstrocytes, neurons and oligodendrocytes and these cell lines are used to model Amyotrophic Lateral Sclerosis and other neurological and neurodegenerative disorders. By assessing the severity of iAstrocyte toxicity in vitro, along with markers of neurodegeneration, the disease progression can be predicted for the patient donor. In addition, the disclosed in vitro methods can be used to subgroup patient populations based on their molecular profile and test whether a patient will react to certain treatment options in order to select the best treatment for them. The disclosed method can also be used to assist in patient selection for clinical trials. The in vitro assays will be complemented with expression analysis (microarrays and RNA sequencing) for a deeper profiling of each cell line. Moreover, epigenetic features of the cells will be assessed to further assess the progression of the neurological disorder or to predict responsiveness of therapeutic treatment.

There are no current assays available to predict disease prognosis or to group patients based on molecular markers or reactivity to a certain drug treatment for most of the neurological and neurodegenerative diseases. One exception is Huntington's disease where genetic testing and age of onset correlates to rate of patient decline. However, gene silencing has the potential to influence the prediction accuracy of disease progression. The innovative aspect is that the disclosed methods use skin cells from the patient to detect markers of the particular neurological or neurodegenerative disease, such as epigenetic markers, morphological markers and functional markers. It is unexpected that skin cells can provide information that is predictive of disease progression of a neurodegenerative disorders using an in vitro assay. The methods disclosed herein which use converted patient skin fibroblasts holds the promise to reveal information on the patient's disease prognosis or on the patient's reactivity to certain treatments.

There is an urgent need for reliable well-defined model systems to improve our understanding of neurological and neurodegenerative disease. In addition, being able to select patient groups for clinical trials will strongly improve the chances to have a positive outcome. Currently, most clinical trials for ALS fail or are not reproducible in different patient cohorts. Since ALS is so diverse, the ability to pre-screen patients for their response to a drug, might be a pre-requisite for successful clinical trials. Until now, the classic reprogramming technology to generate induced pluripotent stem cells (iPSCs) from patient skin biopsies and their consecutive differentiation into NPCs and thereafter iAstrocytes or other cell types, is very time and labor intensive. Therefore, iPSing is not optimally suited to test a potential therapy in real-time for decision taking during enrollment for a clinical trial for a fast progressing disease like ALS 3.

The disclosed methods are quicker and do not require testing of cells that have undergone clonal selection. Instead of producing stem cells, NPCs are made directly within one week after fibroblast derivation. Therefore, this in vitro test is an interesting tool for selecting patients for a clinical trial. Expression data demonstrated that the iAstrocytes generated by this method are highly similar to adult human laser captured spinal cord iAstrocytes. This disclosed model system can help to shed light on the individual roles of several affected cell types and improve our understanding of disease mechanisms. Because the maintenance of these cells is less time consuming, it is possible to grow a large selection of different lines at the same time, allowing a direct comparison between individual patients under equal conditions. The disclosed methods will further attempt to sub-group ALS patients according to reactivity to therapeutic SOD1 reduction in preparation of a clinical trial. This information will be crucial to establish a more reliable screening of patients for the planned clinical trial targeting SOD1.

Conversion of Skin Cells to iNeurons and iAstrocytes

Direct conversion has several major advantages compared to classical reprogramming: 1) no clonal selection; 2) speed and efficiency—it takes only one week to generate iNPCs from a patient fibroblast line and the efficiency is higher than classical iPSing; 3) easy and time saving handling of the iNPCs; 4) instead of starting from pluripotent stem cells, NPCs are generated directly, allowing to skip the first differentiation step (stem cells to NPCs) while still offering the option of generating several cell types affected by ALS. It was recently demonstrated that ALS iAstrocytes and other iNPC-derived cells derived by this new method affect survival of co-cultured mouse MNs in a similar manner as post-mortem spinal cord derived patient astrocytes. Moreover, gene expression profiling of four skin derived astrocyte lines revealed that these cells are highly similar to postmortem laser captured adult human spinal cord astrocytes. Oligodendrocytes generated from ALS patient skin fibroblasts with either traditional reprogramming or direct conversion expressed major oligodendrocyte markers and had equally detrimental effects on co-cultured MNs.

Astrocyte differentiation from iNPCs takes only one week and is performed by seeding the iNPCs in medium containing 10% FBS and 0.2% N2. The oligodendrocyte differentiation has a duration of one month. Briefly, iNPCs are grown in neurobasal, 2% B27 and 15 ng/ml PDGFaa for 2 weeks, then at week three PDGFaa is reduced to 10 ng/ml and IGF-1 is added to the medium (20 ng/ml). During the last week of differentiation IGF-1 concentration is increased to 50 ng/ml and PDGFaa is removed.

Both, astrocyte and oligodendrocyte differentiation can be initiated every time the iNPCs are split, which is twice a week, or the frozen portionated stocks can be used for the standard controls. The production of three consecutive batches of astrocytes from a certain iNPC line for the performance of three replicated independent co-culture experiments takes less than two weeks, while for the oligodendrocytes, about 1.5 months need to be considered.

### Cell Types

The disclosed methods can be carried out on any cells type known to contribute to various neurological and neurodegenerative disorders (neurons, oligodendrocytes and neuronal progenitor cells (NPCs)). For example, neuronal progenitor cells have the capacity to differentiate into Schwann cells and other neuronal cell types including GABAergic, dopaminergic, serotonergic, cholinergic and glutamatergic neurons. These cell types are implicated in many additional neurodegenerative diseases including Alzheimer's Disease, Amyotrophic Lateral Sclerosis (ALS), Parkinson's Disease, Batten Disease forms (CLN1-13), SMA with respiratory distress and Charcot-Marie-Tooth Disease 2S (CMT2S), both caused by mutations in the IGHMBP2 gene, Rett syndrome, Pitt Hopkins Syndrome, NEDAMSS, CLN disease or Batten Disease, NEDAMSS, Huntington's Disease, Fronto-temporal Dementia and Multiple Sclerosis. Furthermore, in some of these disorders, astrocytes contribute to the disease phenotype. In some cases, the contribution of astrocytes and oligodendrocytes to neuronal disease may not induce neuronal death, but they may impact the morphology, function or neuronal physiology.

The disclosed methods allow for better prediction of patient prognosis following diagnosis. Moreover, being able to test patient reactivity to treatment options is vital for the patient to make informed decisions regarding treatment options as well as for potentially planning end of life care. This tool is likely to be used for patient stratification and inclusion/exclusion criteria for clinical trials/drug treatment options similarly to what is current practice with genetic tests in certain kinds of cancer and asthma.

### Subjects

In exemplary embodiments of the present disclosure, the subject is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes). In some aspects, the mammal is a human.

In exemplary aspects, the subject comprises skin cells which may be used to grow primary fibroblasts which may be reprogrammed (e.g., by way of a direct conversion method) to iNPCs, which in turn can differentiate into iAstrocytes and/or neurons and/or oligodendrocytes, and the iAstrocytes and/or neurons and/or oligodendrocytes so obtained in exemplary aspects exhibit elevated levels of basal mitochondrial respiration, mitochondrial basal and/or ATP-linked respiration, or a combination thereof. Methods of measuring levels of basal mitochondrial respiration and mitochondrial basal and/or ATP-linked respiration in cells are known in the art. See, e.g., the EXAMPLES herein.

In exemplary aspects, the subject has or exhibit mitochondrial changes (e.g., changes in mitochondrial function, relative to a control, e.g., elevated levels of basal mitochondrial respiration, elevated mitochondrial basal and/or ATP-linked respiration, or a combination thereof) or mitochondrial dysfunction as evidenced by iAstrocytes and/or neurons and/or oligodendrocytes derived from iNPCs which are in turn derived from skin cells of the subject, or in neurons derived directly from fibroblasts and/or neurons of the subject. In various aspects, the subject is in need of improved or increased neuron survival, reduced basal and/or ATP-linked respiration, reduced oxidative stress (e.g., oxidative stress linked to mitochondrial dysfunction), or a combination thereof. In exemplary embodiments, the subject has elevated or dysfunctional levels of peroxynitrite.

By “mitochondrial dysfunction” is meant a deviation from healthy individuals. In other cases, the mitochondria might show abnormal phenotype such as disturbance of the mitochondrial network or abnormal localization which results in mitochondrial dysfunction. This could also include changes in the cellular metabolism that can influence the mitochondrial activity including the electron transport chain.

The term “ATP-linked respiration or mitochondrial ATP-linked respiration” refers to the process in the mitochondria used to produce energy in the form of ATP. This occurs by sending electrons through an electron transport chain in the inner mitochondrial membrane, which produces a proton gradient across the membrane. The protons are then used by the ATP synthase to produce energy (ATP). This reaction consumes oxygen (ergo, respiration).

By “basal respiration” or “basal mitochondrial respiration” is meant the amount of oxygen consumed by the mitochondria within a cell without any chemically induced manipulation. It is the resting oxygen consumption rate of mitochondria within a given cell type.

By “survival of neurons” or “survival of motor neurons” is meant the ability of a neuron, e.g., motor neuron, to live despite potentially adverse conditions. Suitable methods of measuring neuron survival, e.g. motor neuron survival, are known in the art. In exemplary aspects, motor neuron survival is calculated 3-4 days following co-culture with human iAstrocytes and/or neurons and/or oligodendrocytes from patients or healthy individuals. In various instances, surviving motor neurons (defined as axon projections over 50 microns) are counted in each condition. The number of motor neurons remaining alive in each condition in various aspects is then normalized to the number of surviving motor neurons in non-diseased control lines. Survival is reported as a percent.

By “oxidative stress” is meant cumulative damage within an individual cell and/or body caused by free radicals that were not neutralized by cellular antioxidant processes. Oxidative stress can cause lipid peroxidation, DNA damage and oxidatively modified proteins. As a consequence, it can induce DNA mutations, damage cellular membranes and alter signaling pathways within the cell, ultimately leading to cellular death or dysfunction. In addition, oxidative damage in the central nervous system may impact cellular proliferation and remodeling, neural plasticity and neurogenesis with consequence on synaptic transmission (Salim, J Pharmacol Exp Ther 360(1): 201-205 (2017)). The impact of oxidative stress on neurons and neuronal support cells (such as astrocytes) leads to neurological phenotypes including seizures, behavioral abnormalities and neuronal death.

Suitable methods of measuring levels of peroxynitrite are known in the art. In exemplary aspects, the level of peroxynitrite is measured by measuring a bi-product, e.g., nitrotyrosine (see, e.g., Rios et al., Nitric Oxide, 3rd ed., Elsevier, pages 271-288 (2017)). Also peroxynitrite reacts with tyrosine residues to form nitrotyrosine. Thus, in exemplary aspects, measurement of nitrated proteins is an indicator of the presence of peroxynitrite. In exemplary aspects, probes that detect peroxynitrite in live cells in vitro are used (Wu et al., Anal Chem 89(20) 10924-10931 (2017)).

In exemplary aspects, the subject has a seizure disorder. As used herein, the term “seizure disorder” is meant a medical condition characterized by episodes of uncontrolled electrical activity in the brain, thus producing symptoms that include two or more seizures. In various aspects, the seizure disorder is epilepsy (aka epileptic seizure disorder), simple partial seizure, benign rolandic epilepsy, catamenial epilepsy, atonic seizure, absence seizure, clonic seizure, tonic seizure, febrile seizure. In various aspects, the subject suffers from focal seizures, temporal lobe seizures, frontal lobe seizures, occipital lobe seizures, parietal lobe seizures, generalized seizures, absence seizures, myoclonic seizures, generalized convulsive seizures, generalized tonic-clonic seizures, symptomatic generalized epilepsy, progressive myoclonic epilepsy, reflex epilepsy. In various instances, the subject suffers from Ohtahara Syndrome, Benign Familial Neonatal seizures, infantile spasms, Dravet Syndrome (SCN1A), Rett Syndrome, Angelman Syndrome, Tuberous Sclerosis, Sturge-Weber Syndrome, Febrile Seizures, Landau-Kleffner Syndrome, Lennox-Gastaut Syndrome, Rasmussen Syndrome, Gelastic Epilepsy, Benign Rolandic Epilepsy, Benign Occipital Epilepsy, Childhood Absence Epilepsy, Juvenile Myoclonic epilepsy, neurodevelopmental disorder with regression, abnormal movements, loss of speech, and seizures (NEDAMSS) or epileptic encephalopathy.

In various aspects, the subject has a channelopathy, neuronal hyper excitability, lysosomal storage disease (e.g., Pompe and Batten Disease forms (CLN1-13)), Facioscapulohumeral Muscular Dystrophy (FSHD), seizure disorders caused by SPATA5 mutations, seizures disorders caused by SMARCAL1 mutations, neurological disorders caused by KIF1A mutations, SCN2A, NEDAMSS (IRF2BPL), SLC6A1, SCN1A, epilepsy and other seizure disorders, Huntington's disease, SMA with respiratory distress and Charcot-Marie-Tooth Disease 2S (CMT2S), Rett syndrome, Huntington's Disease, Fronto-temporal Dementia, and Multiple Sclerosis, or a combination thereof. In various instances, the subject has a neurodegenerative disorder associated with mitochondrial dysfunction, such as a neurodegenerative disorder associated with elevated levels of basal and/or ATP-linked respiration. In exemplary aspects, the subject does not have ALS. Optionally, the subject does not suffer from Parkinson's Disease or Alzheimer's Disease. In exemplary aspects, the subject has a disease in which oxidative stress plays a role. In various aspects, the subject has FSHD.

### Neurodegenerative Disease

In exemplary aspects, the neurodegenerative disease is a disorder of the nervous system. In some examples, the neurodegenerative disease involves mitochondrial dysfunction (e.g., elevated levels of basal mitochondrial respiration, elevated mitochondrial basal and/or ATP-linked respiration, or a combination thereof).

In exemplary aspects, the neurodegenerative disease is a neurodegenerative disorder associated with mitochondrial dysfunction, such as a neurodegenerative disorder with elevated levels of basal and/or ATP-linked respiration,

In various instances, the neurodegenerative disease is a disorder of the nervous system wherein cells of the nervous system comprise SCN2A mutations, mutated gene products of the SCN2A gene, the SCN1A gene, the IRF2BPL gene or SLC6A1 gene.

The neurodegenerative disease in various aspects is Alzheimer's disease, Parkinson's disease, Multiple Sclerosis, Amyotrophic Lateral Sclerosis (ALS), other demyelination related disorders, senile dementia, subcortical dementia, arteriosclerotic dementia, AIDS-associated dementia, or other dementias, a central nervous system cancer, traumatic brain injury, spinal cord injury, stroke or cerebral ischemia, cerebral vasculitis, epilepsy, Huntington's disease, Rett Syndrome, SMARD1/CMT25, Tourette's syndrome, Guillain Barre syndrome, Wilson disease, Pick's disease, neuroinflammatory disorders, encephalitis, encephalomyelitis or meningitis of viral, fungal or bacterial origin, or other central nervous system infections, prion diseases, cerebellar ataxias, cerebellar degeneration, spinocerebellar degeneration syndromes, Friedreichs ataxia, ataxia telangiectasia, spinal dysmyotrophy, progressive supranuclear palsy, dystonia, muscle spasticity, tremor, retinitis pigmentosa, striatonigral degeneration, mitochondrial encephalo-myopathies, neuronal ceroid lipofuscinosis such as Batten Disease, hepatic encephalopathies, renal encephalopathies, metabolic encephalopathies, toxin-induced encephalopathies, and radiation-induced brain damage.

### Amyotrophic Lateral Sclerosis (ALS)

ALS is a devastating neurodegenerative disorder whose clinical features are determined by the progressive and inexorable degeneration of motor neurons. The heterogeneity in disease progression suggests the presence of modifying factors, either genetic or environmental that could eventually help to discover new therapeutic strategies that are urgently needed.

In the past decade it has become clear that non-neuronal cells are also affected by the pathogenic mechanisms in ALS and they seem to play a crucial role leading to MN death. The exact roles of these other cell types during the different disease stages remain unclear to date. The current findings from mouse models and published human data from us and others highlight the strong contribution of non-neuronal cells to most ALS disease cases. While MNs are determining the disease onset, oligodendrocytes were implicated in modifying both, disease onset and progression and astrocytes mainly disease progression in various independent studies.

If astrocytes and oligodendrocytes indeed can impact the variability of disease progression, they represent a highly valuable target for future therapeutics and potentially for sub-grouping of ALS patients. Even more since in the clinical setting, the patients are only diagnosed after disease onset and occurrence of evident symptoms

Hallmarks of ALS are the sporadic nature, the great heterogeneity in age of onset, duration of disease and age of death (4). The average disease duration from diagnosis to death is 2-5 years, however, extremely aggressive or, on the opposite, mild variants where death follows as rapidly as 6 months or as late as 20 years after diagnosis, exist. In fact, even patients carrying mutations in the same gene or affected siblings from the same family can present very diverse disease characteristics (5). These are major challenges for understanding the disease mechanisms, the development of new therapeutic strategies and the design of clinical trials 6. Patients with an aggressively progressing disease might not respond to the same treatment as such with milder disease courses. In addition, patients in different stages of the disease might display varying reactions to the same treatment. At the moment, it is not possible to clearly determine disease stages in ALS on a cellular level. Moreover, the course of disease progression of a patient is unknown at the time of enrollment into a clinical trial. A better classification could drastically change the recruiting process for clinical trials and facilitate both, basic research as well as drug development for ALS.

The ALS cases are grouped into two different categories, sporadic (sALS) and familial (fALS). Around 10% of ALS cases are classified as fALS with a predominantly autosomal dominant pattern of inheritance, while the remaining 90% occur sporadically. Among the fALS cases, about 20-25% are caused by mutations in the gene encoding for the superoxide dismutase 1 (SOD1) (Rosen et al. 1993). The analysis of spinal cord tissue obtained from both sALS and fALS patients revealed increased glial activation (Haidet-Phillips et al. 2011). Moreover, it was also reported that the neurotoxic effect promoted by sALS patient-derived astrocytes over MNs was similar with that exerted by fALS astrocytes (Haidet-Phillips et al. 2011; Meyer et al. 2014). These findings support that the glial-based ALS pathogenesis could be transversal between different forms of the disease. However, little is known about the profile and gene/protein expression of these glial cells, as well as the mechanisms by which they become neurotoxic.

Increasing evidence supports that microRNAs (miRNAs) have an important role in the neuroinflammatory and neurodegenerative mechanisms linked to ALS (Sharma and Lu 2018; Volonte et al. 2015). However, the common use of microarrays might prevent the detection of important alterations and miRNA deregulation for disease pathology due to the limitation in the detection of mature miRNAs due to their small size (Chugh and Dittmer 2012). In particular, some miRNAs have been defined as inflamma-miRs since they are associated with innumerous inflammatory pathways (Tahamtan et al. 2018), and from them, miR-155, miR-146a and miR-21 as an important trio (Olivieri et al. 2013; Quinn and O'Neill 2011). Increased expression of these three miRNAs was found in the lumbar spinal cord of symptomatic SOD1G93A mice, while reduced levels of miR-146a and miR-21 were observed in the brain cortex (Cunha et al. 2018; Gomes et al. 2019). Interestingly, a discriminatory signature of miR-155/miR-21/miR-146a being specifically upregulated in spinal mSOD1 astrocytes and downregulated in cortical ones was observed. Moreover, it was described that miRNAs can be transferred from cells into extracellular vesicles (EVs) and shuttled to recipient cells (Fernandes et al. 2018; Pinto et al. 2017; Prada et al. 2018).

Batten Disease

In exemplary aspects, the neurodegenerative disorder is caused by a defect in lysosomal storage clearance process which results in the accumulation of large molecules within cells, also known as a lysosomal storage disease. For example, the neurodegenerative disorde is neuronal ceroid lipofuscinosis (CLN), also referred to Batten disease. Such CLN disorders include mutation in the CLN1 gene, CLN2 gene, CNL3 gene, CLN4 gene, CLN5 gene, CLN6 gene, CLN7, CLN8 or CLN10.

In particular, there are at least 67 disease causing-mutations in the CLN3 gene have been described. However, 85% of patients are homozygous for the 1.02 kb deletion leading to the loss of exon 7 and exon 8. CLN3 mutations found in patients predominantly cause reduced abundance or functionality of the protein (battenin).

SCN2A Mutations

In exemplary, the subject comprises a SCN2A mutation or a mutated SCN2A gene product, e.g., an SCN2A mRNA or SCN2A protein. The SCN2A gene is known in the art as the sodium voltage-gated channel alpha subunit 2 gene, and also as HBA; NAC2; BFIC3; BFIS3; BFNIS; HBSCI; EIEE11; HBSCII; Nav1.2; SCN2A1; SCN2A2; Na(v)1.2. The SCN2A gene sequence can be found at NCBI accession number NC 000002.125. The SCN2A gene encodes the alpha subunit of this voltage-gated sodium channel transmembrane glycoprotein. SCN2A is located in the human genome at ch. 2q24.3 and has 27 confirmed exons (suggested exons not confirmed=31). The sequences of the alpha subunit of isoforms 1 and 2 are listed in the NCBI database as follows.

| | Protein | | mRNA | |
|---|---|---|---|---|
| | Accession | SEQ | Accession | SEQ |
| | No. | ID NO: | No. | ID NO. |
| Isoform 1 | NP_001035232.1 | 1 | NM_001040142.2 | 2 |
| Isoform 2 | NP_001035233.1 | 3 | NM_001040143.2 | 4 |

The SCN2A mutation may be any one of those SCN2A mutations described in the art. See, e.g., Shi et al., Brain Dev. 34(7): 541-545 (2012), Sanders et al., Trends in Neurosciences 41(7): 442-456. The SCN2A mutation may also be a new mutation that is currently not described. In exemplary aspects, the SCN2A mutation is a deletion, insertion, substitution mutation in the SCN2A gene. In various aspects, the SCN2A mutation is a missense mutation or a microduplication. In exemplary aspects, the SCN2A mutation is a nonsense mutation, synonymous mutation, silent mutation, neutral mutation, duplication mutation, splice mutation, or point mutation. Exemplary gene mutations are described in Mandieh and Rabban, Iran J Pedatr 23(4): 375-388 (2013). In some aspects, the gene mutation occurs in Exon 1, Exon 2, Exon 3, Exon 4, Exon 5, Exon 6, Exon 7, Exon 8, Exon 9, Exon 10, Exon 11, Exon 12, Exon 13, Exon 14, Exon 15, Exon 16, Exon 17, Exon 18, Exon 19, Exon 20, Exon 21, Exon 22, Exon 23, Exon 24, Exon 25, Exon 26, Exon 27 or a combination thereof (suggested exons not confirmed =31). In some aspects, the mutation could be in an intron, altering the splicing of the SCN2A mRNA leading to inclusion or exclusion of any of the exons described above or to the activation of a cryptic splice site that leads to the insertion of intronal sequences into the mRNA. In exemplary aspects, the mutation is any one of those listed in Table A. In exemplary instances, the mutated SCN2A gene product comprises a deletion, insertion, or substitution mutation in the SCN2A gene product. For instance, the mutated gene product may be a mutated SCN2A mRNA comprising a nucleic acid deletion, nucleic acid insertion, or nucleic acid substitution mutation relative to the wildtype SCN2A mRNA sequence. The SCN2A mRNA contains 27 exons (26 are coding) that encode a 2005 amino acid protein (called Nav1.2) (suggested exons not confirmed=31). In various aspects, the mutated gene product may be a mutated SCN2A protein comprising an amino acid deletion, amino acid insertion, or amino acid substitution relative to the wildtype. In various aspects, the mutation occurs in Domain I, Domain II, Domain III, or Domain IV of the protein encoded by the SCN2A gene. In various aspects, the mutation occurs in the extracellular domain, transmembrane domain, or intracellular domain of the protein. In various aspects SCN2A amino acid sequence, the mutation is a nonsense, canonical splice sites, frameshift insertion/deletions or large deletion in the first 1591 amino acids or the first 4773 nucleotides. In various aspects, the mutation is a nonsense, canonical splice sites, frameshift insertion/deletions or large deletion within the C-terminal portion of the amino acid sequence (e.g., a portion of the amino acid sequence starting at the amino acid at position 1592 to the C-terminal amino acid). In some aspects, the mutation is a protein truncation or a gene duplication. In exemplary aspects, the subject comprises a SCN2A-mediated disorder, such as any one of those described in Sanders et al., Trends in Neurosciences 41(7): 442-456, e.g., infantile epileptic encephalopathy (IEE), characterized by infantile-onset seizures, before 12 months of age, followed by neurodevelopmental delay; benign (familial) infantile seizures (BIS), characterized by infantile-onset seizures, before 12 months of age, that resolve by 2 years of age without overt long-term neuropsychiatric sequelae; and autism spectrum disorder/intellectual disability (ASD/ID), characterized by global developmental delay, particularly of social and language milestones. In various aspects, the SCN2A-mediated disorder is epileptic encephalopathy with choreoathetoid movements, benign infantile seizures with late-onset episodic ataxia, childhood-onset epileptic encephalopathy, and schizophrenia. In some aspects, SCN2A mutations could also lead to additional neurological phenotypes such as depression, avoidance of stimuli, reduced visual capacity.

TABLE A

| Chr2 | | | |
|---|---|---|---|
| Nucleotide (s) | Ref | Alt | Reference |
| 166164448 | G | A | 10 |
| 166152578 | A | G | 9 |
| 166170231 | G | A | 9 |
| 166170231 | G | A | 9 |
| 166183403 | — | A | 9 |
| 166201312 | G | A | 9 |
| 166245137 | A | T | 9 |
| 166152367 | G | A | 9 |
| 166201311 | C | T | 9 |
| 166201379 | C | A | 9 |
| 166210819 | G | T | 9 |
| 166231378 | T | C | 9 |
| 166234111 | C | T | 9 |
| 166198975 | G | A | 3 |
| 166198975 | G | A | 3 |
| 166245184 | C | A | 7 |
| 166231247 | T | C | 6 |
| 166234116 | A | G | 4 |
| 166179821-166179822 | CT | — | 1 |
| 166172100 | — | A | 1 |
| 166201311 | C | T | 1 |
| 166231415 | G | A | 2 |
| 166243265 | C | T | 5 |
| 166187838 | A | G | 8 |

In exemplary aspects, the subject has an SCN2A-mediated disorder such as any of those described in Sanders et al., 2018, supra and Wolff et al., Brain 140(5):1316-1336 (2017). Mouse models for SCN2A mutations have been described, for example, see Kearney et al., Neuroscience. 2001; 102(2):307-17 (incorporated by reference in its entirety).

### SLC6A1 Mutations

In various exemplary aspects, the subject comprises a SLC6A1 mutation or a mutated SLC6A1 gene product, e.g., a SLC6A1 mRNA or SLC6A1 protein. The SLC6A1 gene encodes a gamma-aminobutyric acid (GABA) transporter (GAT1) and alteration in GAT1 leads to aberrant tonic GABA inhibition, which results in absence seizures in GAT-1 knockout mice (Cope et al., Nat Med 2009; 15:1392-1398). The SLC6A1 gene sequence can be found at NCBI Gene ID: 6529 (NC 000003.12). SLC6A1 mutation has been associated with early onset absence epilepsy. Exemplary gene mutations include, but are not limited to, A288V, R44Q, L151Rfs*35, W193×, G457Hfs*10 or G234S. In related embodiments the subject may have epileptic encephalopathy. Mouse models for SLC6A1 mutations have been described, for example, see Madsen et al., J Pharmacol Exp Ther. 2011 July; 338(1): 214-219 and Xu et al., Biochem Biophys Res Commun. 2007 Sep. 21; 361(2):499-504 (incorporated by reference in their entirety). Any of these models may be used to investigate the methods or treatment disclosed herein.

The SLC6A1 mutation may be any one of those SLC6A1 mutations described in the art. See, e.g., Johannesen et al., Epilepsia. 2018 February;59(2):389-402. The SLC6A1 mutation may also be a new mutation that is currently not described. In exemplary aspects, the SLC6A1 mutation is a deletion, insertion, substitution mutation in the SLC6A1 gene. In various aspects, the SLC6A1 mutation is a missense mutation or a microduplication. In exemplary aspects, the SLC6A1 mutation is a nonsense mutation, synonymous mutation, silent mutation, neutral mutation, duplication mutation, splice mutation, or point mutation. In some aspects, the gene mutation occurs in Exon 1, Exon 2, Exon 3, Exon 4, Exon 5, Exon 6, Exon 7, Exon 8, Exon 9, Exon 10, Exon 11, Exon 12, Exon 13, Exon 14, or Exon 15. In some aspects, the mutation could be in an intron, altering the splicing of the SLC6A1 mRNA leading to inclusion or exclusion of any of the exons described above or to the activation of a cryptic splice site that leads to the insertion of intronal sequences into the mRNA. In exemplary aspects, the mutation is any one of those listed in Table B.

TABLE B
SLC6A1 mutation

c.104dupA p.Lys36GluFsTer171
c.223G > A p.Gly75Arg
c.419A > G p.Tyr140Cys
c.434C > T p.Ser145Phe
c.578G > A p.Thp193Ter
c.695G > T, p.Gly232Val
c.809T > C p.Phe270Ser
C.863C > T p.Ala288Val
c.881_883del p.Phe294del
C.987C > A p.Cys329Ter
c.1024G > A p.Val342Met
C.1070C > T p.Ala357Val
c.1084g > a p.Gly362Arg
C.1155C > G p.Phe385Leu
c.1342A > T p.Lys448Ter
c.1369_1370 delGG Gly457HisFsTer10
de novo
C.1377C > G p.Ser459Arg
C.1531G > A p.Val511Met
C.1600C > T p.Gln534Ter
c.850-2A > G
c.6, 1528-1G > C
3p25.3 del.
including SLC6A11 and SLC6A1 (exon 1)

### SCN1A Mutations

In various exemplary aspects, the subject comprises a SCN1A mutation or a mutated SCN1A gene product, e.g., a SCN1A mRNA or SCN1A protein. The SCN1A mutation may be any one of those SCN1A mutations described in the art (For example, see SCN1A mutations in Parihar et al., Journal of Human Genetics volume 58, pages573-580 (2013), which is incorporated by reference in its entirety). The SCN1A gene encodes the alpha subunit of voltage-gated sodium channel Nav1.1. This sodium channel is found on the surface of nerve cells and is essential for the generation and transmission of electrical signals in the brain. The SCN1A gene is also known as GEFSP2, HBSCI, NAC1, Nav1.1, SCN1, sodium channel protein, brain I alpha subunit, sodium channel, voltage gated, type I alpha subunit, sodium channel, voltage-gated, type I, alpha, sodium channel, voltage-gated, type I, alpha polypeptide, or sodium channel, voltage-gated, type I, alpha subunit. The SCN1A gene sequence can be found at NCBI Gene ID: 6323 (NC 000002.12). SCN1A mutation has been associated with genetic (generalized) epilepsy with febrile seizures Plus (GEFS+) and Dravet syndrome (DS, severe myoclonic epilepsy of infancy) (Essay and Goldin, Epilepsia. 2010 September; 51(9): 1650-1658). The SCN1A mutation may be any one of those SCN1A mutations described in the art. See, e.g., Parihar et al., Journal of Human Genetics 58, pages 573-580 (2013), which is incorporated by reference in its entirety. The SCN1A mutation may also be a new mutation that is currently not described. Mouse models for SCN1A mutations have been described, for example, see Kang et al., Epilepsia Open. 2019; 4(1): 164-169 and Miller et al., Genes Brain Behay. 2014 February;13(2):163-72 (incorporated by reference in their entirety). Any of these models may be used to investigate the methods or treatments of disclosed herein.

### Interferon Regulatory Factor 2 Binding Protein Like (IRF2BPL)

In various exemplary aspects, the subject comprises an interferon regulatory factor 2 binding protein like (IRF2BPL) mutation or a mutated IRF2BPL gene product, e.g., an IRF2BPL mRNA or IRF2BPL protein. The IRF2BPL gene encodes a member of the IRF2BP family of transcriptional regulators (Marco Gliese et al., Am J Hum Genet. 2018 Aug. 2; 103(2):245-260). The IRF2BPL gene is also known as C14or f4, EAP1, or NEDAMSS. The IRF2BPL gene sequence can be found at NCBI Gene ID: 64207 (NC 000014.9). The disease that has been associated with IRF2BPL mutations includes Neurodevelopmental Disorder With Regression, Abnormal Movements, Loss Of Speech, And Seizures (NEDAMSS). The IRF2BPL mutation may be any one of those IRF2BPL mutations described in the art. See, e.g., Marcogliese et al., Am J Hum Genet. 2018 Aug. 2; 103(2):245-260; Tran Mau-Them et al., Genetics in Medicine 21, pages1008-1014(2019); Shelkowitz et al., Parkinsonism Relat Disord. 2019 62:239-241; Shelkowitz et al., Am J Med Genet A. 2019 November; 179(11):2263-2271. which are all incorporated by reference in their entirety. The IRF2BPL mutation may also be a new mutation that is currently not described. In exemplary aspects, the mutation is any one of those listed in Table C (IRF2BPL mutations described in Marcogliese et al., Am J Hum Genet. 2018 Aug. 2; 103(2):245-260 and Tran Mau-Them et al., Genetics in Medicine 21, pages1008-1014(2019)).

TABLE C
IRF2BPL mutation

p.Glu172*
p.Gln127*
p.Arg188*
p.Pro372Arg
p.Lys418Asn
chr14: g.77493617G > C −
NM_024496.3: c.519C > G

### Rett Syndrome

Rett syndrome (RTT) is an X linked neurodevelopmental disorder affecting approximately 1 in 10,000 girls. Patients exhibit vast mutation and disease heterogeneity. The onset is typically characterized by the loss of previously achieved developmental milestones at 6-18 months of age with a progressive loss of motor function and cognitive function 2. In 95% of typical Rett syndrome cases, the disease is caused by deficiency of the transcription factor methyl-CpG-binding protein 2 (MeCP2), a key regulator of gene expression in the central nervous system (CNS). MeCP2 functions as an important epigenetic reader 6, transcriptional activator, and repressor of thousands of genes in the central nervous system with regional and cell type specific alterations in gene expression. Underlying clinical phenotypes of RTT is a global neuronal phenotype featuring compaction of neurons characterized by smaller soma and shortened and fewer neurites. Furthermore, clinically, and animal modeling has shown a direct connection between disease severity and neuroanatomical changes dependent on various MeCP2 mutations.

### Pitt Hopkins Syndrome

In any of the methods, subject has a mutation in the gene encoding Transcription factor TCF4 (alias ITF2, SEF2 or E2-2) that results in impaired or reduced function of TCF4 protein. Missense, nonsense, frame-shift and splice-site mutations as well as translocations and large deletions encompassing TCF4 gene have been shown to cause Pitt-Hopkins syndrome (PTHS).

The TCF4 gene (MIM #610954) is located on chromosome 18q21.2, and it has 20 exons (the first and the last are noncoding) that span 360 kb. This transcription factor is a broadly expressed basic helix-loop-helix (bHLH) protein that functions as a homo- or heterodimer. The TCF4 exhibits transcription-regulatory activities that is highly expressed during early human development throughout the central nervous system, the sclerotome, peribronchial and kidney mesenchyme, and the genital bud, playing an important role in cellular proliferation, lineage commitment, and cellular differentiation.

Several alternatively spliced TCF4 variants have been described, allowing for the translation of at least 18 protein isoforms, with different N-terminal sequences. The following are exemplary mutations of the TCF4 gene known to cause PTHS: whole gene deletions, such as large rearrangements that are several megabases in size, partial gene deletions, such as deletions involving one or more of the exons from 7 to 20, balanced translocations, such as deletions disrupting the coding sequence of the gene, missense mutations, such as deletions involving the bHLH domain of TCF4, nonsense and frameshift mutations, such as mutations spread throughout the gene between exons 7 and 18, and slice site mutations, such as those affecting the donor and acceptor consensus splice sites and those that result in the shift off the reading frame. Exemplary genomic mutations include t(14;18)(q13.1;q21.2) and t(2;18)(q37;q21.2), which are de novo balanced translocations, respectively, with breakpoints falling within the second half of the gene. Additional exemplary mutations include: c.1486+5 g>T, c.520C>T(p.Arg174×), c.1726C>T, c.624de1c (p.Arg576×), C.624de1c, and c.550-2A>G.

### Treatment

As used herein, the term “treat,” as well as words related thereto, do not necessarily imply 100% or complete treatment. Rather, there are varying degrees of treatment of which one of ordinary skill in the art recognizes as having a potential benefit or therapeutic effect. In this respect, the methods of treatment of the present disclosure can provide any amount or any level of treatment. Furthermore, the treatment provided by the method of the present disclosure can include treatment of one or more conditions or symptoms or signs of the cancer being treated. Also, the treatment provided by the methods of the present disclosure can encompass slowing the progression of the disease, disorder or medical condition aimed for treatment. For example, the methods can treat a neurodegenerative disease by virtue of enhancing cognitive and/or motor ability, reducing tremors, reducing muscle stiffness, improve balance, decrease amnesia, enhance speech ability, and the like. In exemplary aspects, the methods treat by way of delaying the onset or recurrence of the disease, disorder, or medical condition, or a sign or symptom thereof, by at least 1 day, 2 days, 4 days, 6 days, 8 days, 10 days, 15 days, 30 days, two months, 3 months, 4 months, 6 months, 1 year, 2 years, 3 years, 4 years, or more.

As used herein, the term “reduced” or “decreased” or synonyms thereof may not refer to a 100% or complete reduction or decrease. Rather, there are varying degrees of reduction or decrease of which one of ordinary skill in the art recognizes as having a potential benefit or therapeutic effect. In exemplary embodiments, the reduction provided by the methods of the present disclosure is at least or about a 10% reduction (e.g., at least or about a 20% reduction, at least or about a 30% reduction, at least or about a 40% reduction, at least or about a 50% reduction, at least or about a 60% reduction, at least or about a 70% reduction, at least or about a 80% reduction, at least or about a 90% reduction, at least or about a 95% reduction, at least or about a 98% reduction) relative to a control. In exemplary embodiments, the decrease provided by the methods of the present disclosure is at least or about a 10% decrease (e.g., at least or about a 20% decrease, at least or about a 30% decrease, at least or about a 40% decrease, at least or about a 50% decrease, at least or about a 60% decrease, at least or about a 70% decrease, at least or about a 80% decrease, at least or about a 90% decrease, at least or about a 95% decrease, at least or about a 98% decrease) relative to a control.

As used herein, the term “elevated” or “increased” or synonyms thereof may not refer to a 100% or complete elevation or increase. Rather, there are varying degrees of elevation or increase of which one of ordinary skill in the art recognizes as having a potential benefit or therapeutic effect. In exemplary embodiments, the increase provided by the methods of the present disclosure is at least or about a 10% increase (e.g., at least or about a 20% increase, at least or about a 30% increase, at least or about a 40% increase, at least or about a 50% increase, at least or about a 60% increase, at least or about a 70% increase, at least or about a 80% increase, at least or about a 90% increase, at least or about a 95% increase, at least or about a 98% increase) relative to a control. In exemplary embodiments, the elevation provided by the methods of the present disclosure is at least or about a 10% elevation (e.g., at least or about a 20% elevation, at least or about a 30% elevation, at least or about a 40% elevation, at least or about a 50% elevation, at least or about a 60% elevation, at least or about a 70% elevation, at least or about a 80% elevation, at least or about a 90% elevation, at least or about a 95% elevation, at least or about a 98% elevation) relative to a control.

As used herein, the term “improve” or “enhance” or synonyms thereof may not refer to a 100% or complete improvement or enhancement. Rather, there are varying degrees of improvement or enhancement of which one of ordinary skill in the art recognizes as having a potential benefit or therapeutic effect. In exemplary embodiments, the improvement or enhancement provided by the methods of the present disclosure is at least or about a 10% improvement or enhancement (e.g., at least or about a 20% improvement or enhancement, at least or about a 30% improvement or enhancement, at least or about a 40% improvement or enhancement, at least or about a 50% improvement or enhancement, at least or about a 60% improvement or enhancement, at least or about a 70% improvement or enhancement, at least or about a 80% improvement or enhancement, at least or about a 90% improvement or enhancement, at least or about a 95% improvement or enhancement, at least or about a 98% improvement or enhancement) relative to a control.

### Diagnostic Methods

The present disclosure also provides methods of identifying a subject who is responsive to a therapy. In exemplary embodiments, the method comprises analyzing iAstrocytes and/or neuron and/or oligodendrocytes generated from iNPCs derived from skin cells obtained from the subject or derived directly from fibroblasts obtained from the subject for a SCN2A mutation, a mutated SCN2A voltage-gated sodium channel protein, subjects with SCN1A mutations, IRF2BPL mutations or SLC6A1 mutation. In various aspects, the method further comprises obtaining skin cells from the subject. In various instances, the method further comprises generating induced neuronal progenitor cells (iNPCs) from skin cells obtained from the subject or generating neurons directly from fibroblasts obtained from the subject. In exemplary aspects, the method further comprises differentiating iNPCs into iAstrocytes and/or neurons and/or oligodendrocytes. In exemplary instances, the skin cells obtained from the subject are used to grow primary skin fibroblasts. Optionally, a direct conversion method is used to produce iNPCs. Such methods are described in Meyer et al., PNAS 829-832 (2014)).

In exemplary embodiments, the method of identifying a subject who is responsive to a therapy comprises analyzing the level of mitochondrial activity or energy state of astrocytes generated from induced neuronal progenitor cells derived from skin cells obtained from the subject, wherein the subject is identified as a subject who is responsive to the therapy when the astrocytes exhibit elevated mitochondrial activity compared to astrocytes from a healthy subject. In various aspects, the method further comprises a step of obtaining skin cells from the subject. In various instances, the method further comprises a step of generating induced neuronal progenitor cells (iNPCs) from skin cells obtained from the subject. In exemplary aspects, the method further comprises differentiating iNPCs into astrocytes or neurons. Optionally, the skin cells obtained from the subject are used to grow primary skin fibroblasts. In various aspects, the mitochondrial activity is analyzed by measuring basal mitochondrial respiration, mitochondrial basal and/or ATP-linked respiration, or a combination thereof, of the astrocytes. In exemplary instances, the energy state is analyzed by measuring oxygen consumption and lactate production or extracellular acidification rate, or a combination thereof of the astrocytes.

As used herein, the term “energy state” means a status of mitochondrial energy metabolism as described in Zhang and Zhang, Methods Mol Biol 1928: 353-363 (2019) and Zhang et al., Nat Protoc 7(6): doi:10.1038/nprot.2012.048. In exemplary aspects, the energy state of astrocytes is determined by measuring the oxygen consumption (OCR) and lactate production (extracellular acidification rate, ECAR) and then plotting the OCR as a function of ECAR to produce an energy map. Suitable methods of measuring OCR and ECAR are known in the art and include, for instance, the protocol described in Plitzko, B. and Loesgen, S. (2018). Bio-protocol 8(10): e2850. DOI: 10.21769/BioProtoc.2850; and Plitzko, B., Kaweesa, E. N. and Loesgen, S. (2017). J Biol Chem 292(51): 21102-21116; and Zhang and Zhang, Methods Mol Biol 1928: 353-363 (2019). In various instances, OCR is a measure of mitochondrial respiration and ECAR is a result of glycolysis.

Further provided herein are methods of treating a subject in need thereof. In exemplary embodiments, the method comprises identifying a subject who is responsive to a therapy in accordance with the presently disclosed identifying methods and administering that therapy to the identified subject. In exemplary embodiments, the method comprises (a) obtaining a skin cell sample from the subject (b) generating iAstrocytes and/or neurons and/or oligodendrocytes from iNPCs derived from skin cells obtained from the subject or generating neurons from fibroblast cells obtained from the subject, (b) analyzing the iAstrocytes and/or neurons and/or oligodendrocytes for a SCN2A mutation, a mutated SCN2A voltage-gated sodium channel protein, a SLC6A1 mutation, a SCN1A mutation or a mutation in IRF2BPL.

Methods of analyzing cells for a mutation are known in the art. In exemplary embodiments, the analysis comprises karyotyping, fluorescence in situ hybridization (FISH), comparative genomic hybridization (CGH), polymerase chain reaction (PCR), Multiplex PCR, Nested PCR, Real-time PCR, Restriction fragment length polymorphism (RFLP); Amplification refractory mutation system (ARMS); RT: Reverse transcriptase; Multiplex ligation-dependent probe amplification (MLPA); Denaturing Gradient Gel Electrophoresis (DGGE); Single Strand Conformational Polymorphism (SSCP); heteroduplex analysis; Chemical cleavage of mismatch (CCM); Protein truncation test (PTT); Oligonucleotide ligation assay (OLA), DNA microarray, DNA sequencing, Next Generation Sequencing (NGS) and the like. See, e.g., Mandieh and Rabbani, 2013, supra. Methods of analyzing cells for a mutated SCN2A voltage-gated sodium channel protein are known in the art. The methods in some aspects include an immunoassay using an antibody specific for the mutation. The immunoassay in various aspects is immunoprecipitation, Western blotting, ELISA, radioimmunoassay, and the like.

The sample in various aspects comprises a skin biopsy, e.g., a skin punch. In various aspects, the skin biopsy is used to grow skin cells such as primary skin fibroblasts.

Also provided herein are methods of determining effectiveness of a therapy. In exemplary embodiments, the method comprises analyzing the level of mitochondrial activity of astrocytes generated from induced neuronal progenitor cells derived from skin cells obtained from the subject after administration of that therapy, wherein a decrease in basal and/or ATP-linked respiration in the astrocytes, or a decrease in oxidative stress in the astrocytes, or increase in surviving neurons cultured on top of pretreated astrocytes as compared to astrocytes from the subject before administration of the is indicative of an effective therapy. In various instances, the method further comprises a step of generating induced neuronal progenitor cells (iNPCs) from skin cells obtained from the subject. In exemplary aspects, the method further comprises differentiating iNPCs into astrocytes or neurons. Optionally, the skin cells obtained from the subject are used to grow primary skin fibroblasts. In various aspects, the mitochondrial activity is analyzed by measuring basal mitochondrial respiration, mitochondrial basal and/or ATP-linked respiration, or a combination thereof, of the astrocytes. In various aspects, effectiveness of a therapy can also be measured using neurons derived from patient skin cells by measuring survival, differentiation efficiency and length of neurites with and without that treatment.

The following examples are given merely to illustrate the present invention and not in any way to limit its scope.

### EXAMPLES

### Example 1

ALS Patient Astrocytes and Oligodendrocytes Mimic Disease Progression

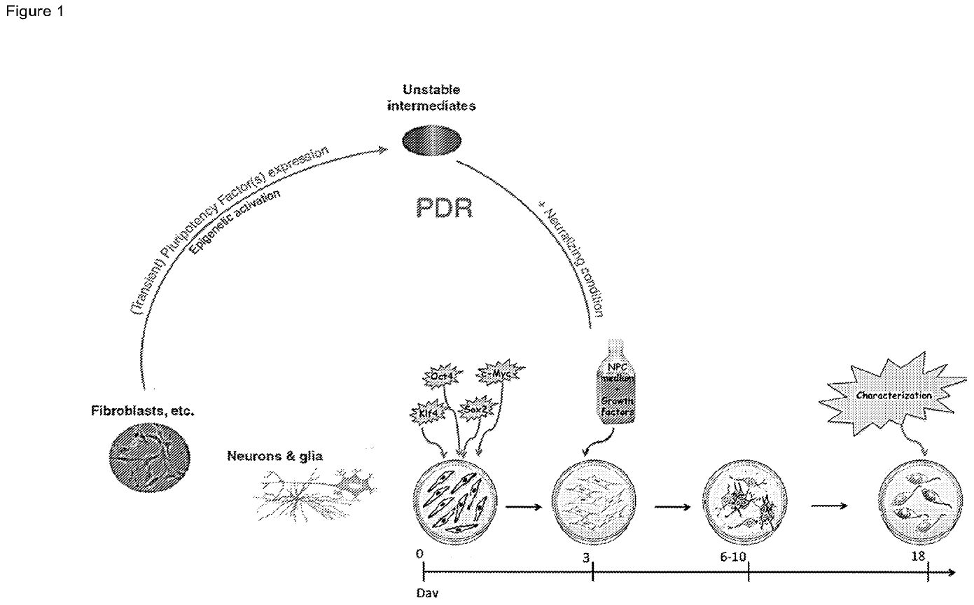

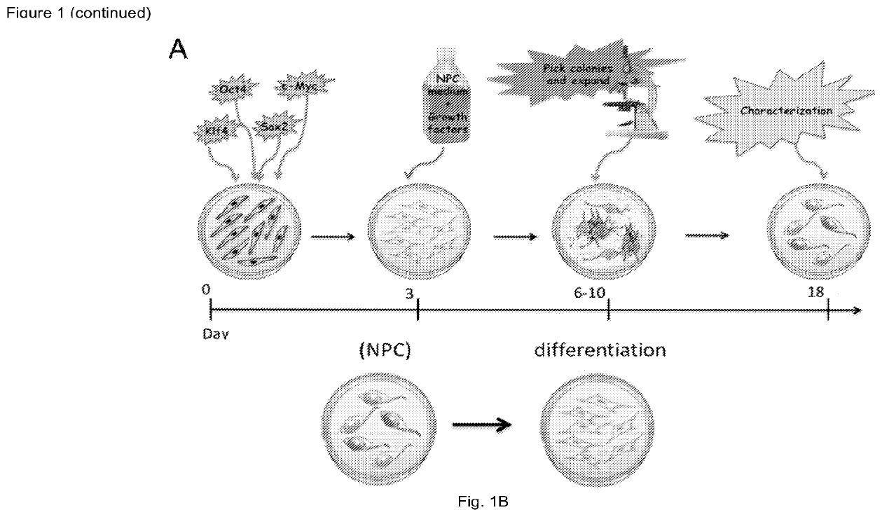

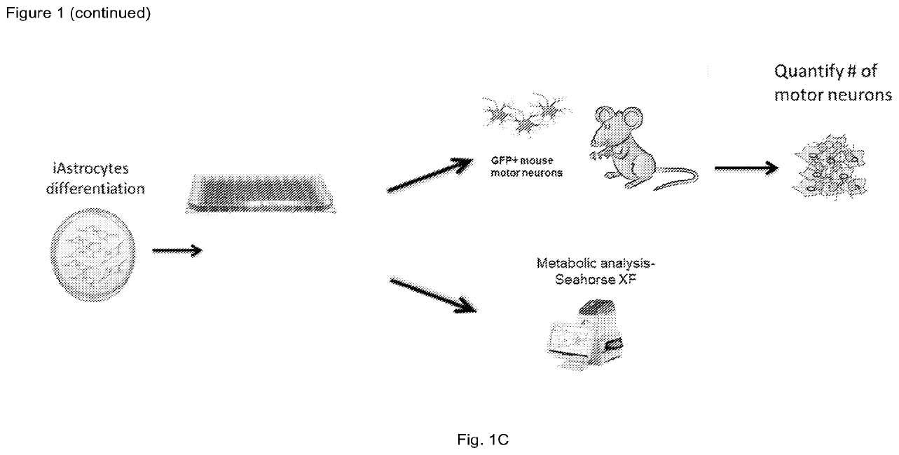

Preliminary data has demonstrated that the impact of skin derived astrocytes and oligodendrocytes from ALS patients on motor neuron survival, varies. rates and within various subtypes (sALS, SOD1, C9ORF) of onset as well as additional controls. The fibroblasts were converted to induced neuronal progenitor cells (iNPCs) as previously described (Meyer 2014, supra) as shown FIG. 1. Neuronal progenitor cells were cultured on fibronectin coated dishes in NPC media (DMEM/F12 media containing 1% N2 supplement (Life Technologies), 1% B27 and 20 ng/ml fibroblast growth factor-2) until confluent. Astrocytes were differentiated by seeding a small quantity of NPCs on another fibronectin coated dish in astrocyte inducing media (DMEM media containing 0.2% N2 and 10% FBS). The converted Astrocytes are referred to iAstrocyte.

To determine if the ALS patient astrocytes are toxic to motor neurons, the iAstrocytes were co-cultures with motor neurons. For the co-culture, stem cells from HB9:GFP+ mouse embryos were cultured as described previously (Meyer 2014). Embryonic bodies (EB) were cultured in EB differentiation media (knockout DMEM/F12, 10% knockout serum replacement, 1% N2, 0.5% L-glutamine, 0.5% glucose, and 0.0016% 2-mercaptoethanol) with smoothen agonist and retinoic acid freshly added starting day 2 of differentiation. EBs were dissociated with papain as previously described (Meyer 2014) and sorted. GFP+ motor neurons were seeded on top of patient iAstrocytes in a 96 well plate (10,000 cells per well) or 384 well (1,000 cells per well). Co-cultures were imaged with InCell 6000 (GE Healthcare) for up to four days. Motor neurons with neurite outgrowth of greater than 50 um were counted as alive. Data was normalized to healthy controls. This experiment demonstrated that ALS iAstrocytes show various degrees of toxicity to neurons.

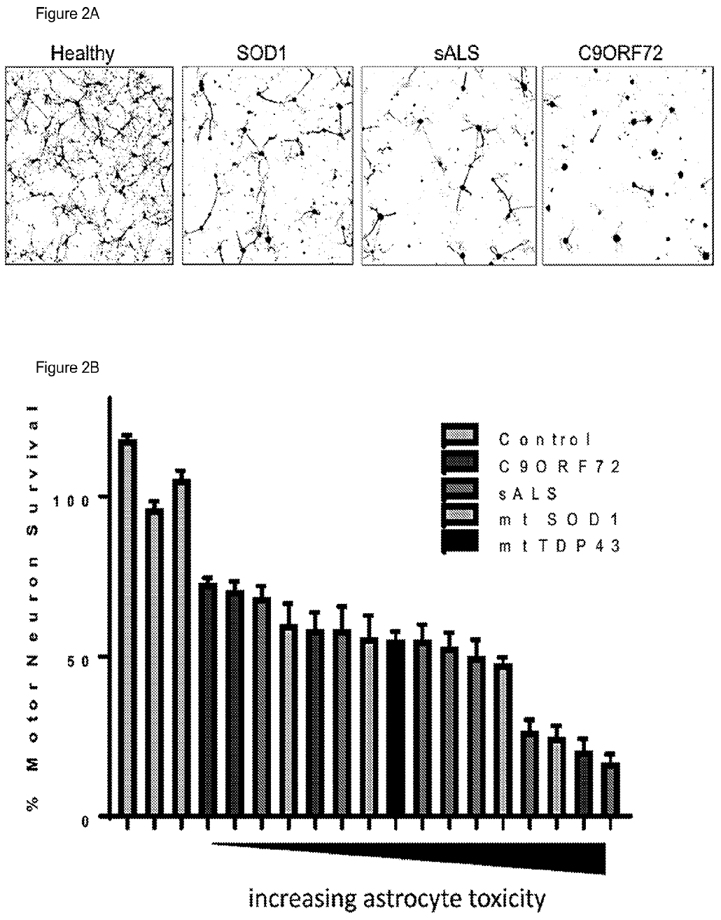

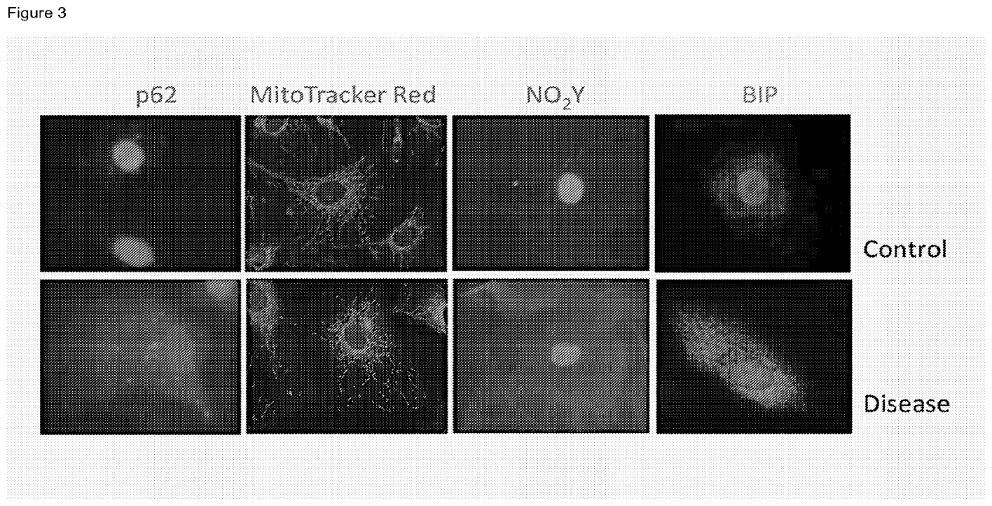

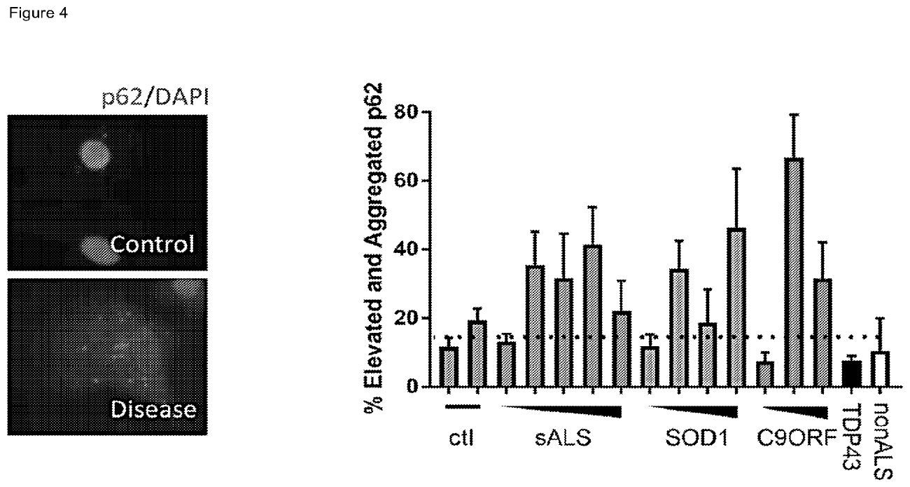

As shown in FIG. 2A, iAstrocytes from ALS patients were toxic to motor neurons The data indicates that this toxicity is independent of disease classification (e.g. C90RF72, sALS, mtSOD1, ect. FIG. 2B). In addition, the ALS patient iAstrocytes display disease markers, such as p62, mitotracker Red and NO₂ and BIP, which are known to have correlate with ALS pathology in postmortem tissue (FIG. 3). The number of cells (%) with elevated p62 and formation of aggregated does not correlate with astrocyte severity (FIG. 4) however may be used in combination with other markers to further subgroup the patient lines.

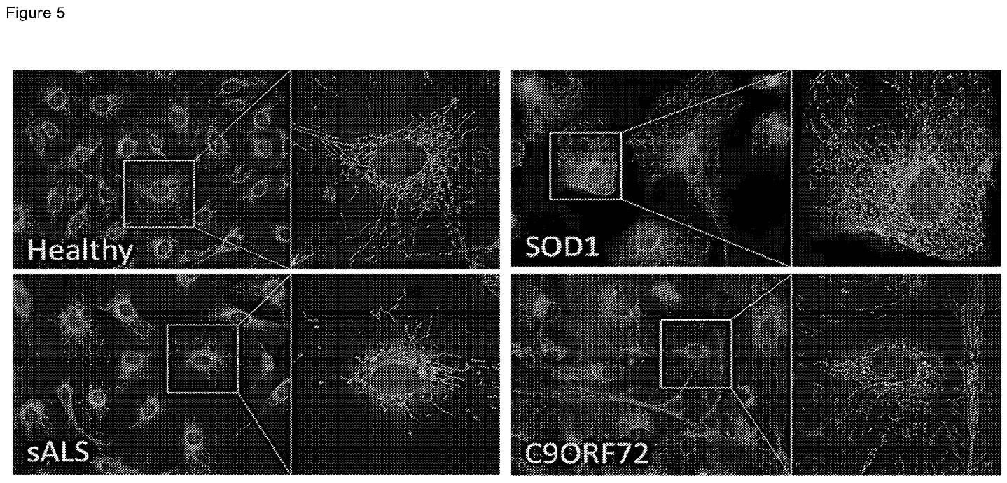

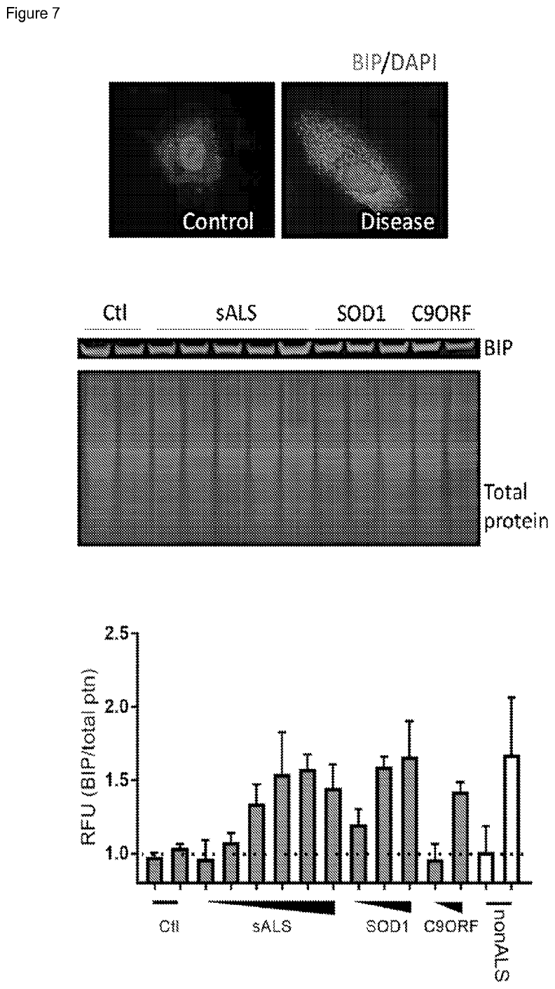

Additional studies include evaluating the reprogrammed cell lines for markers of cellular stress. Mitochondrial morphology and ER stress in the ALS patient derived cells are evaluated by immunostaining for BIP and comparing these stains to age matched healthy controls (FIG. 3). Immunostaining results are further confirmed by performing western blots for BIP. BIP levels are normalized against a total protein loading control, prior to a second normalization to healthy control lines (see FIG. 7). Variation in mitochondrial morphology, such as abnormal rounded mitochondria and fragmented networks, varies in severity between ALS disease subpopulations (see FIG. 5) and there was a correlation between astrocyte aggression and the number of astrocytes with rounded mitochondria.

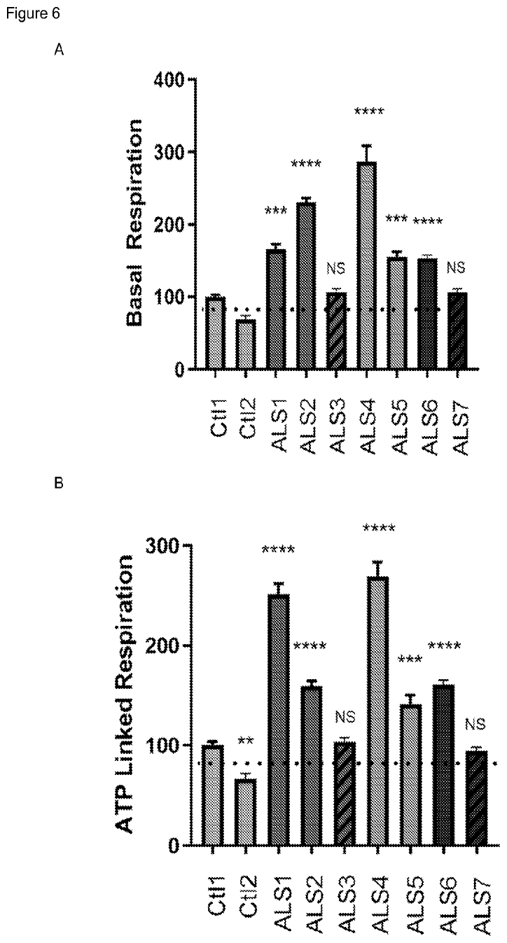

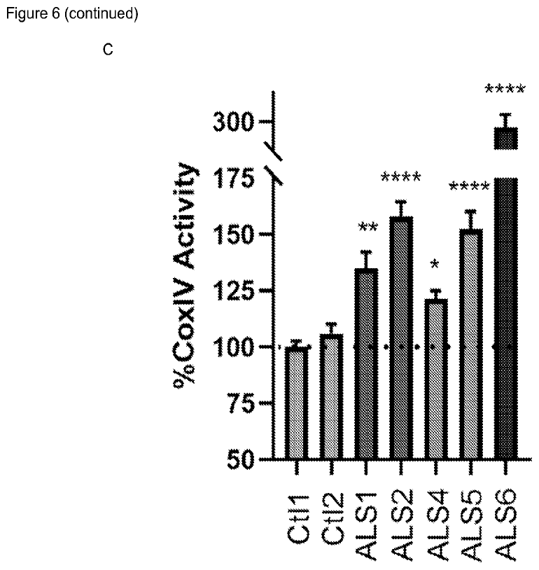

Further, the mitochondrial activity of iAstrocytes from ALS patients was evaluated. Induced astrocytes were seeded on 24 well seahorse plates in quadruplicate or a 96 well seahorse plate in quintuplicate. Twenty-four hours later media was replaced with seahorse base media containing 10Mm glucose and 2Mm glutamine. Oligomycin (1 Um), FCCP (5Um) and antimycin A (10Um) were injected separately to evaluate oxygen consumption following inhibition of the ATPase synthase, mitochondrial uncoupling and total shutdown of the electron transport chain. Alternatively, iAstrocyte media was replaced with a mitochondrial buffer (220Mm mannitol, 70Mm sucrose, 10Mm KH2PO4, 5Mm HepesHopes, 1Mm EGTA and 0.2% Fatty acid free BSA) to measure complex IV dependent oxygen consumption. CoxIV activity was induced using 0.5Mm TMPD, 2Mm Ascorbate, 1Mm ADP, 10Um antimycin A, 10Mm azide and 1.1 nM seahorse XF, membrane permeabilizer. Oxygen consumption was measured in both assays using the Seahorse XF and Seahorse XFe. As shown in FIG. 6A-6C, ALS patient cell lines have differential levels of basal, ATP-linked respiration and CoxIV activitywhich can be further used to subgroup patients lines, .

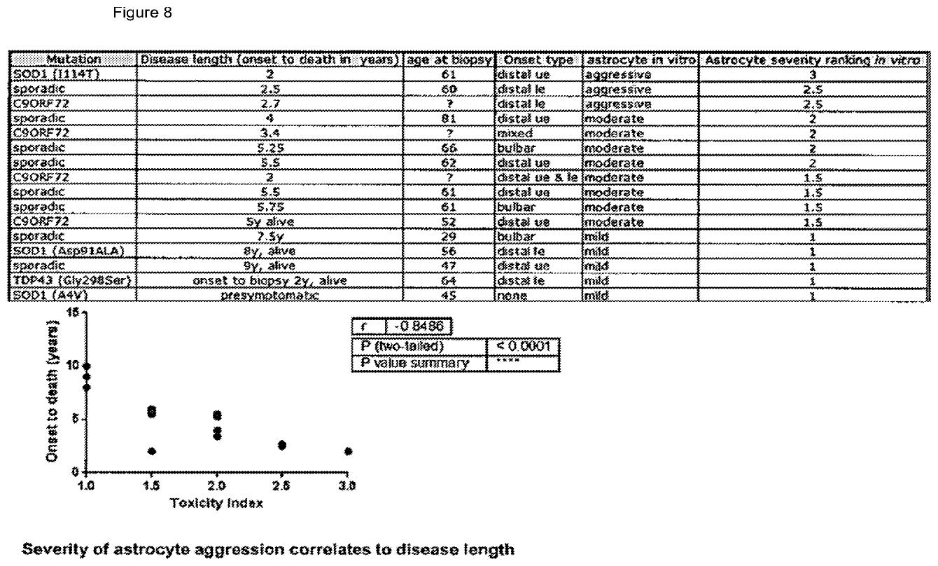

FIG. 8 provides a list of the iAstrocyte cell lines converted from fibroblasts obtained for ALS patients. The degree of toxicity (referred to as toxicity index) of each iAstrocyte cell line on motor neurons was determined using the co-culture assay described above. This analysis demonstrates that the severity of the toxicity correlates with disease length.

### Example 6

Mitochondrial Respiration of iAstrocytes

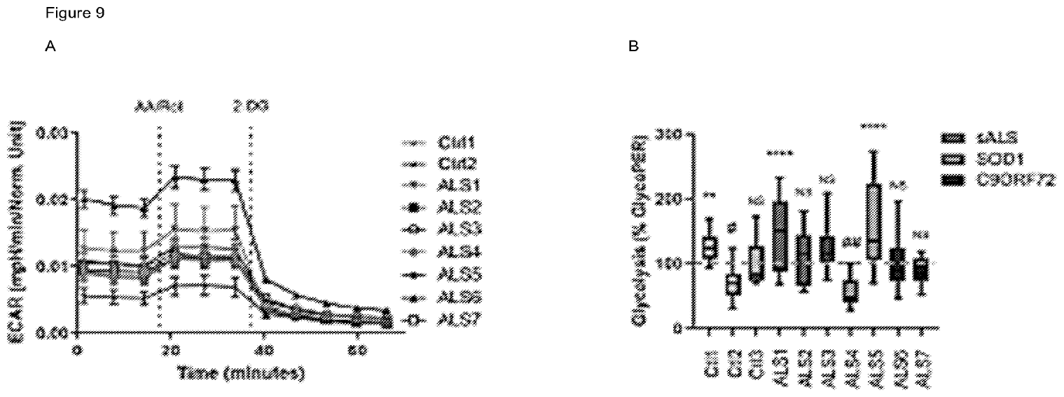

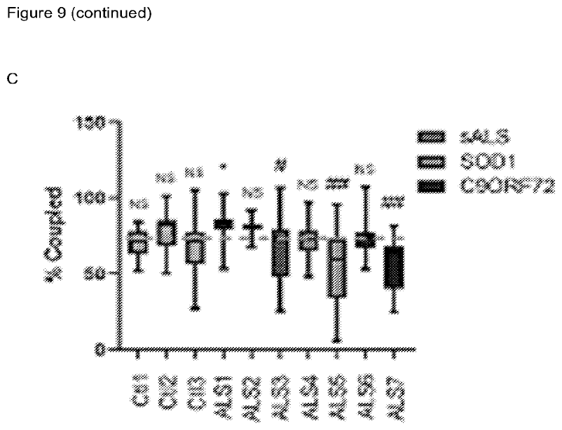

While variation in ALS disease markers is common amongst patients and animal models, one of the most shared abnormalities is dysregulated energy metabolism (Dupuis et al., Lancet Neurol. 2011; 10(1):75-82). Given that iAstrocytes are primarily glycolytic, cellular glycolysis in patient iAstrocytes were measured by extracellular flux analysis and a representative rate graph is shown (FIG. 9A). Only ALS1 and ALS5 had significantly elevated glycolysis where as ALS4 had a significant reduction when compared to healthy controls (FIGS. 9A and 9B) demonstrating that glycolysis could be used to subgroup patient cell lines.

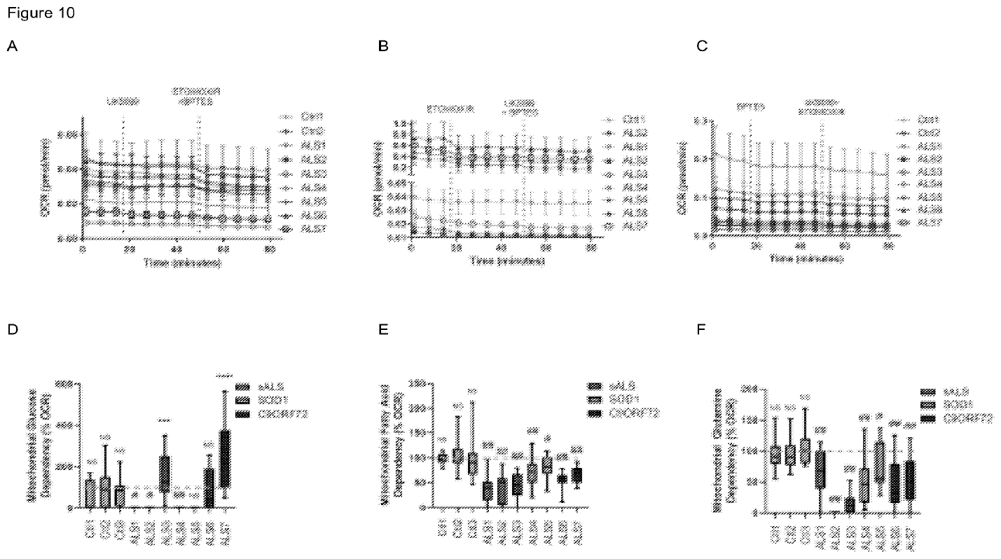

Changes in mitochondrial dependency on different fuel sources may explain the variation in oxidative phosphorylation observed between patient lines. Thus, mitochondrial dependency on glucose, glutamine and long chain fatty acids as fuel sources was examined by extracellular flux analysis and representative rate graphs are shown (FIGS. 10A-C). A significant decrease in the mitochondrial dependency of all ALS iAstrocytes on glutamine and fatty acids as well as a differential dependency on glutamine was observed (FIG. 10D). These results suggested that the patient lines seem to have more flexibility in the use of different fuel sources than healthy controls which is another parameter that may be used to subgroup the patient lines.

### Example 3

Misfolded SOD1 Levels as a Marker to Help Identifying Patient Sub-Groups for Clinical Trials

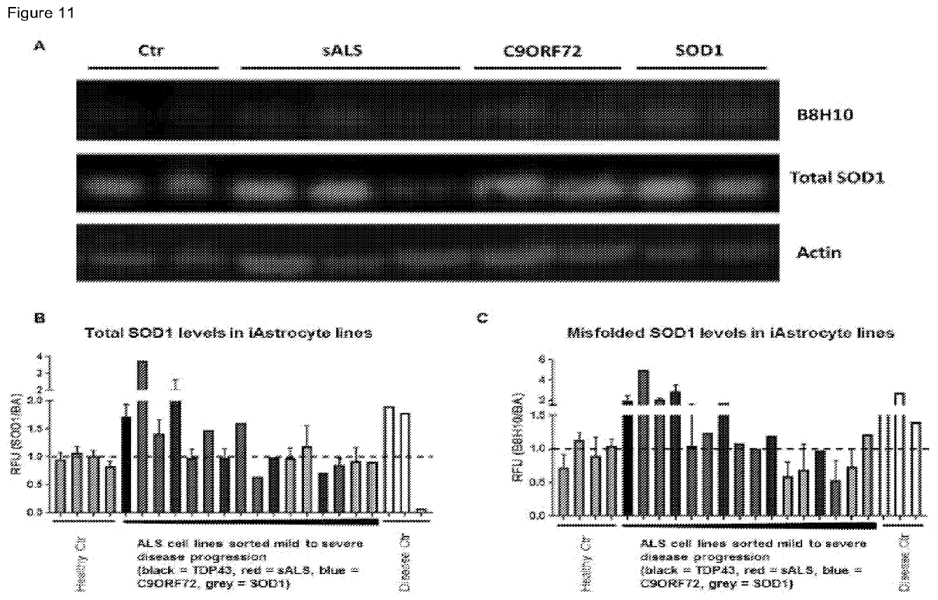

The level of misfolded SOD1 levels in the ALS patient cell lines compared to healthy controls as well as disease controls. Antibodies against total SOD1 protein, as well as misfolded SOD1 (B8H10, C4H6) were used to detect protein expression by immunofluorescent stainings, as well as western blots (FIG. 11A). Western blot quantification demonstrated variable levels of total SOD1 protein in cell lines from ALS patients.

A trend towards higher levels of total SOD1 protein abundance in patient cell lines that had a less severe impact on motor neuron survival in co-culture compared to the severe cell lines (FIG. 11B). Moreover, this was also true when probing the same cell lysates with the B8H10 antibody for misfolded SOD1 with even clearer distribution towards higher signal in cell lines that have a milder impact on motor neuron health (FIG. 11C). Elevated levels of total SOD1 as well as misfolded SOD1 in the disease control samples from an adult patient with a mild neuropathy, a patient suffering from Spinal Muscular Atrophy and a patient with neuropathy carrying a mutation in the properdin gene. Of note, none of these disease control iAstrocytes display an effect on motor neuron survival.

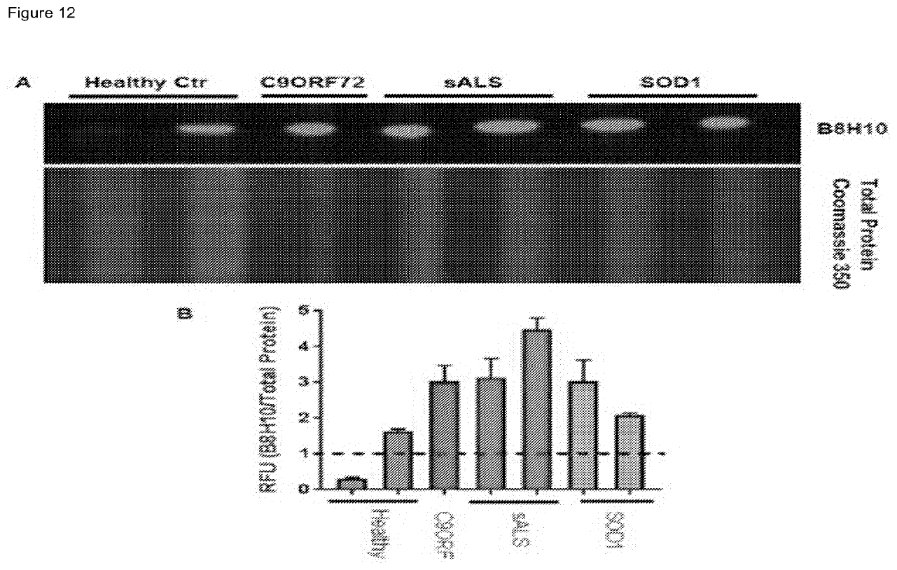

The data for the cell lines from patients with severe disease progression were re-evaluating by normalizing to total loaded protein instead using Coomassie R350. This protein stain fluoresces when imaged with the licor system. Moreover, we also established native page immunoblot protocols using a milder lysis protocol and non-denaturing gels. This method uses a milder lysis protocol and preserves correct folding of SOD1 dimers. Using this method and comparing to total loaded protein instead of actin, the misfolded SOD1 signal was 2-4 fold higher compared to healthy control samples in all samples tested so far (FIG. 12). Importantly, the misfolded SOD1 that was detected in this assay migrated at different location compared to dimeric and monomeric forms of SOD1, which further supports the specificity of this assay for misfolded SOD1.

### Example 4

iAstrocytes from ALS Patients have Decreased Expression of miR-146a

miR-146A is a negative-feedback regulator of the iAstrocyte mediated inflammatory response. It was of interest to investigate whether iAstrocytes from ALS patients have decreased expression of miR-146a. The fibroblasts were converted to induced neuronal progenitor cells (iNPCs), and these neuronal progenitor cells were differentiated to iAstrocytes as described in Example 1. The patients are summarized in the Table 1 below.

TABLE 1

| | | | Age at | | Symptoms | | |
|---|---|---|---|---|---|---|---|
| | | | biopsy, | | onset to | | |
| Cell line | Diagnosis | Mutation | yrs | Onset type | biopsy | | Sex |
| CTR 1 | Non-ALS | — | 42 | — | — | | Male |
| CTR 2 | Non-ALS | — | 64 | — | — | | Female |
| ALS 1 | fALS | SOD1 | 46 | Unknown | 3 | months | Not specified |
| ALS 2 | fALS | SOD1 | 63 | Unknown | 8 | months | Female |
| ALS 3 | fALS | SOD1^{A4V} | 40 | Unknown | Unknown | | Male |
| ALS 4 | sALS | Unknown | 29 | Bulbar | 2.7 | years | Male |
| ALS 5 | sALS | Unknown | 62 | Distal upper extremity | 2.5 | years | Female |
| ALS 6 | sALS | Unknown | 47 | Distal upper extremity | 1.75 | years | Female |
| ALS 7 | sALS | Unknown | 69 | Distal upper extremity | 2.25 | years | Female |

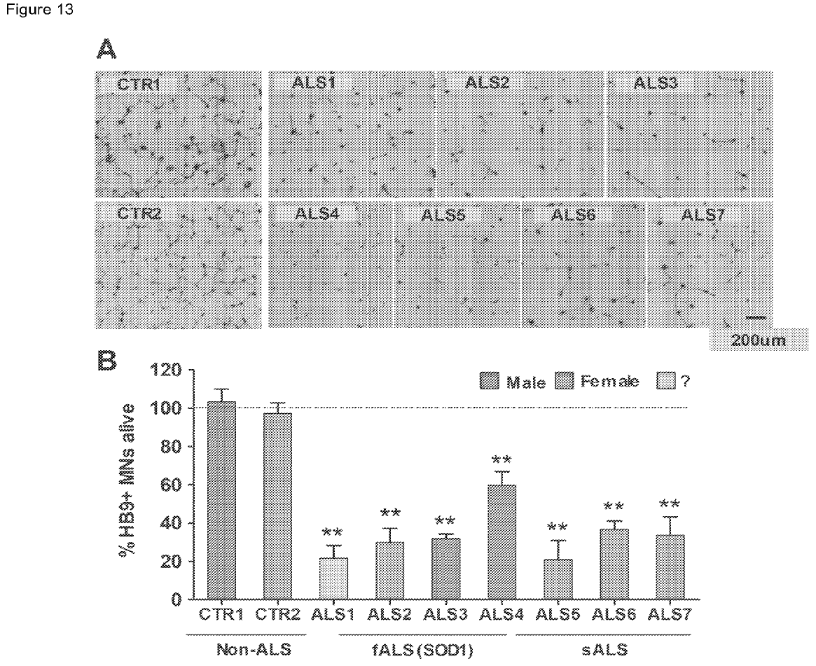

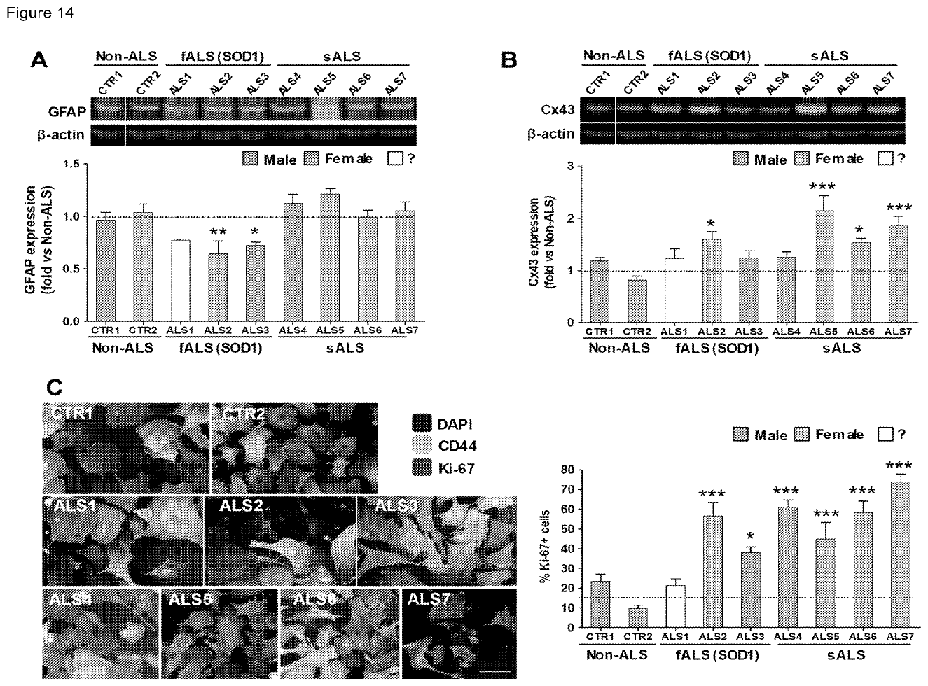

Astrocytes derived from both fALS and sALS patients have previously shown toxicity towards MNs (Haidet-Phillips et al. 2011; Meyer et al. 2014) and transplantation of sALS astrocytes from induced pluripotent stem cells (iPSCs) caused in vivo neuronal degeneration (Qian et al. 2017). Therefore, the neurotoxic potential of the converted cells from ALS patients as described in Example 1. iAstrocytes isolated from both fALS and sALS, independently of gender, were able to cause a reduction in the number of HB9-positive cells after coculturing (p<0.01, FIGS. 13A,B). Interestingly, iAstrocytes derived from ALS4 line (sALS patient) revealed to be the less neurotoxic, when compared with the other cell lines. These results demonstrated that both fALS- and sALS-derived iAstrocytes determined secretome-mediated degeneration of MNs. Decreased expression of GFAP was observed in 1 male and 1 female fALS (ALS2 and ALS3), without significant alterations in the remaining fALS (ALS1) and all sALS cell lines (FIG. 14). Its downregulation was shown to accelerate disease progression in SOD1H46R mouse model (Yoshii et al. 2011) and may be a feature of specific SOD1 mutations, considering that GFAP upregulation usually associates with astrocyte proliferation and reactive astrogliosis (Sofroniew and Vinters 2010).

Connexin-43 (Cx43) is also a marker of astrocyte reactivity that was found to be upregulated in the cortical astrocytes isolated from mS0D1 mice (Gomes et al. 2018), as well as in ALS i PSC-derived astrocytes, which showed impact on MN survival (Almad et al. 2016). As shown in FIG. 14B, Cx43 was increased in all female iAstrocytes (at least p<0.05). Furthermore, since astrocyte proliferation is also a feature associated with astrogliosis and astrocyte aberrancy, Ki-67 expression was assessed in the ALS iAstrocytes. As shown in FIG. 14C, the iAstroctyes were double stained for Ki-67 (red, proliferation marker) and CD44 (green, astrocyte marker), and IN CELL developer and analyzer software to automatically count the number of Ki-67 positive cells. Except fALS1, all cells exhibited increased Ki-67 pattern suggestive of elevated proliferative index (at least p<0.05, FIG. 14C). Therefore, data validate the existence of astrocyte aberrant markers in most of sALS- and fALS iAstrocytes, though fALS2 and fALS3 samples were those reflecting conjoint deregulation of GFAP, Cx43 and Ki-67. Since miRNA deregulation was also shown to be linked with astrocyte reactivity and dysfunction, either promoting astrocyte reactivity or exacerbating their inflammatory response, mi-RNA representation in iAstrocytes from control and patient iAstrocytes cell lines was investigated.

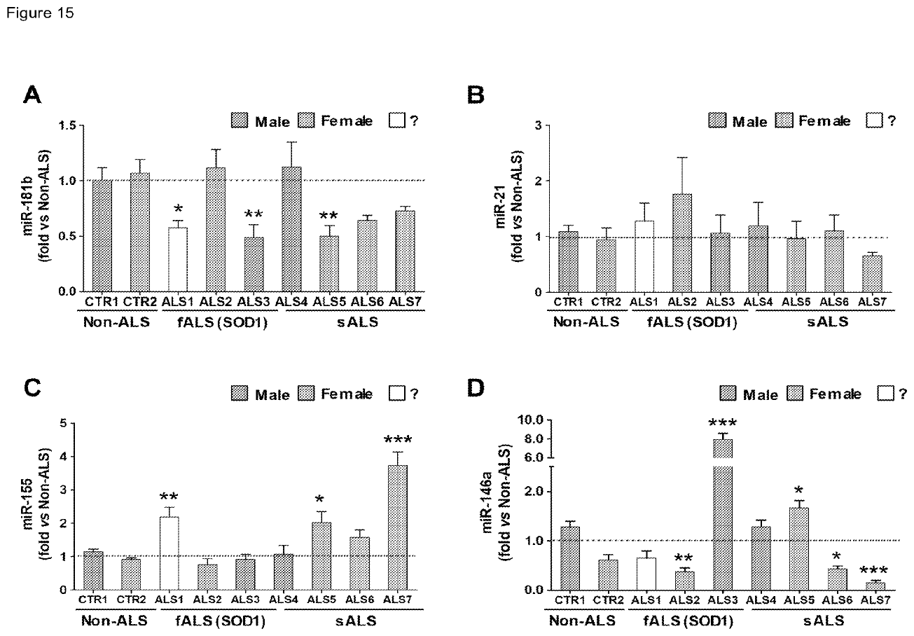

Dysregulated miRNAs in both fALS and sALS cases were shown to be involved in neuroinflammatory and neurodegenerative pathways (Emde et al. 2015; Figueroa-Romero et al. 2016; Volonte et al. 2015). Specifically, miR-181b was found enriched in astrocytes compared to neurons and indicated to suppress the production of oxidative/pro-inflammatory proteins in astrocytes (Hutchison et al. 2013). Here, the reduced expression of miR-181b in 3 iAstrocytes patient cell lines (fALS1, fALS2 and sALS5 (at least p<0.05, FIG. 15A) may be associated with a particular reactive fingerprint, namely in the fALS2 (GFAP^{low}/Cx43^{high}/Ki67^{high}) and in sALS5 (Cx43^{high}/Ki67^{high}) cells. Though inhibition of mi R-21 was demonstrated to ameliorate excessive astrocyte activation following optic nerve crush (Li et al. 2018) and to regulate astrocytic response following spinal cord injury (Bhalala et al. 2012), no changes were observed (FIG. 15B). To notice, however, that miR-155 was also elevated in both sALS1 and fALS5 (at least p<0.05, FIG. 15C), apart sALS7 (p<0.001), reinforcing the proinflammatory signature of these ALS-specific iAstrocytes. Actually, increased levels of miR-155 were previously identified in the mS0D1 mice model and ALS patients (Butovsky et al. 2015; Cunha et al. 2018), and in microglia, such miR-155 upregulation is associated to inflammatory induction, which evolves to a transition state with low levels of miR-155 and miR-146a upregulation (Su et al. 2016). Therefore, inconsistent pattern of up- and down-regulated miR-146a in our patient ALS-iAstrocytes lines (FIG. 15D) suggests that cells may undertake either reparative (fALS3 and fALS5, at least p<0.05), or inflammatory (fALS2, sALS6 and sALS7, at least p<0.05) responses, respectively. Because secreted miRNAs are transported by extracellular vesicles (EVs) and participate in cell-to-cell communication influencing the function of recipient cells (Gaudet et al. 2018), we decided to next evaluate the cargo of such selected inflammatory-associated miRNAs in small EVs released from controls and patient iAstrocytes

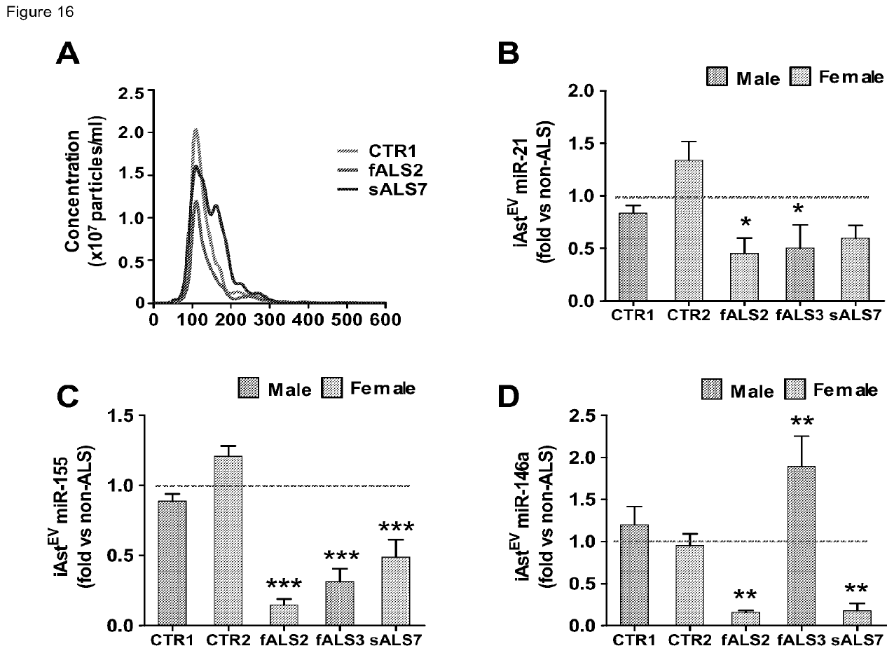

Representation of unaltered miR-21 and deregulated miR-155 and miR-146a in ALS-iAstrocytes was investigated in small EVs released from fALS2 (GFAPlow/Cx43^{high}/Ki67^{high}) fALS3 (GFAPlow/Ki67^{high}/miR-181blow/miR-146alow) and sALS7 (Cx43^{high}/Ki67^{high}/miR-155^{high}/miR-146a1ow), which were selected as representing the most notorious aberrancies, including opposite miR-146a signals. Small EVs are membrane-bound vesicles, with an average size around 50-150 nm, released by most cells into the extracellular environment and suggested to be significant players and carriers of miRNAs in cell-to-cell communication (Barile and Vassalli 2017; Yuan et al. 2018). The small EVs released by iAstrocytes were isolated by differential ultra-centrifugation as described in Cuhna et al. (2016). Briefly, after 24 h incubation in medium with exosome-depleted FBS, cell supernatant was centrifuged at 1000 g for 10 min to pellet cell debris. Then, the supernatant was transferred to another tube and centrifuged at 16000 g for 1 h to pellet microvesicles. After that, the recovered supernatant was filtered in a 0.22 μm pore filter and further centrifuged in an Ultra L-XP100 centrifuge (Beckman Coulter Inc., California, USA) at 100 000 g for 2 h to pellet small EVs (size −100 nm). The pellet of small EVs was then washed in PBS and centrifuged again at 100 000 g for 2 h. All centrifugations were performed at 4° C. The size distribution and concentration of small EVs were assessed by Nanoparticle tracking analysis (NTA) using the Nanosight, model LM10-HSBF (Malvern, UK) and the NTA software version 3.1. The characterization of iAstrocyte-derived small EVs by NTA showed a diameter size ranging from −80 to 200 nm, with a central pick at −100 nm (representative nanoparticle tracking analysis measurements are depicted in FIG. 16A).

miR-155 (p<0.001), but also miR-21 and miR-146a were depleted in EVs released by almost all the selected patient iAstrocytes lines (FIG. 16B-D), what suggests a restricted dissemination from cell-to-cell. Similar depleted representation of inflammatory-associated miRNAs was observed in EVs from iPSCs-derived astrocytes from Alzheimer's disease patients. Exception was observed for mi R-146a that recapitulated the high levels found in fALS3 iAstrocytes (at least p<0.01). Decrease of mi R-146a in both fALS2 and fALS7 astrocytes, and its recapitulation in their derived small EVs, was previously noticed in our study in the brain cortex of SOD1G93A mice at pre-symptomatic and symptomatic stages, as well as in spinal and cortical astrocytes (Gomes et al. 2018), validating mi R-146a as a potential therapeutic target in some patients. The same is true for miR-21 and miR-155 in iAstrocyte-derived small EVs indicating that the controlled enrichment of those miRNAs in EVs by bioengineering may have therapeutic potential in both fALS and sALS cases.

### Example 5

Correlation Between iAstrocyte In Vitro Toxicity and Clinical Prognosis

Fibroblasts were obtained from 9 ALS patients. The fibroblasts were converted to induced neuronal progenitor cells (iNPCs) as previously described (Meyer 2014, supra). Neuronal progenitor cells were cultured on fibronectin coated dishes in NPC media (DMEM/F12 media containing 1% N2 supplement (Life Technologies), 1% B27 and 20 ng/ml fibroblast growth factor-2) until confluent. iAstrocytes were differentiated by seeding a small quantity of NPCs on another fibronectin coated dish in iAstrocyte inducing media (DMEM media containing 0.2% N2 and 10% FBS). The converted iAstrocytes are referred to iAstrocyte.

The iAstrocytes from ALS patients carrying SOD1, C9or f72 or sporadic patients were assessed for 5 different pathological hallmarks of ALS as well as their toxicity against motor neurons, i.e. the cell type that degenerates in ALS. The hallmarks included p62, SOD1, TDP43, abnormal mitochondria, or glutamate.

It was observed that patient iAstrocyte toxicity against motor neurons showed a strong correlation with clinical prognosis. The biomarkers presented the most severe impairment in the most toxic patient astrocyte lines and, when considered as a panel, the most toxic patient astrocytes are the ones displaying the highest number of dysregulated biomarkers. Expression of the biomarker tested correlated with the severity of the impairment observed in the pathological hallmarks as a whole, and in particular p62 accumulation. Increased expression of one or more biomarkers correlated with a negative prognosis of ALS as demonstrated by a shorter clinical onset to death in the patient. This data is summarized in Table 2 below.

TABLE 2
Pathological markers assessed in ALS patients and scored from most severe (***), moderately
severe (**), least severe (*), least mild (+), moderately mild (++), most mild (+++).

| Pathological markers | Pat 1 | Pat 2 | Pat 3 | Pat 4 | Pat 5 | Pat 6 | Pat 7 | Pat 8 | Pat 9 |
|---|---|---|---|---|---|---|---|---|---|
| p62 | 75 | 59 | 64 | 32 | 36 | 59 | 52 | 39 | 41 |
| | *** | * | ** | +++ | +++ | * | + | ++ | ++ |
| SOD1 | 0.06 | 1.44 | 0.78 | 0.91 | 0.5 | 1.4 | 0.49 | 1.76 | 0.43 |
| | +++ | ** | + | * | ++ | ** | ++ | *** | ++ |
| TDP43 | 2.5 | 0 | 7 | 1.5 | 7 | 2.5 | 1.5 | 0 | 0 |
| | * | +++ | *** | + | *** | * | + | +++ | +++ |
| Mitochondria | 1.7 | 1.5 | 1.3 | 1 | 1 | 1 | 1 | 1 | 1 |
| | *** | ** | * | +++ | +++ | +++ | +++ | +++ | +++ |
| glutamate | 34 | 10 | 19 | 13 | 16 | 5 | 11 | 7 | 10 |
| | *** | + | ** | * | ** | +++ | + | +++ | * |
| clinical onset to death (months) | 27 | ? | 19.4 | 31.7 | 21 | 72 | 90 | Alive | Alive |
| | * | | *** | + | *** | ++ | +++ | +++ | +++ |
| In vitro toxicity | 3 | 3 | 3 | 2 | 2 | 1 | 1 | 1 | 1 |
| | *** | *** | *** | + | + | +++ | +++ | +++ | +++ |

### Example 6

Characterization of iAstrocytes from Patients with SCN2A Mutations

Fibroblasts were obtained from 5 patients having SCN2A mutations. The fibroblasts were converted to induced neuronal progenitor cells (iNPCs), and these neuronal progenitor cells were differentiated to iAstrocytes as described in Example 1. The cell lines are referred to as: patient cell lines S4, 5, 1104. 038 and 048 and are described in Table 3 below.

TABLE 3

| Cell Line | ID | Mutation |
|---|---|---|
| 542 | Ctl | N/A |
| 155 | Ctl1 | N/A |
| Ag | Ctl2 | N/A |
| S3 | Ctl3 | N/A |
| S4 | SCN2A-1 | LOF |
| S5 | SCN2A-2 | LOF |
| 1104 | SCN2A-3 | GOF |
| 038 | SCN2A-4 | |
| 048 | SCNA- | |

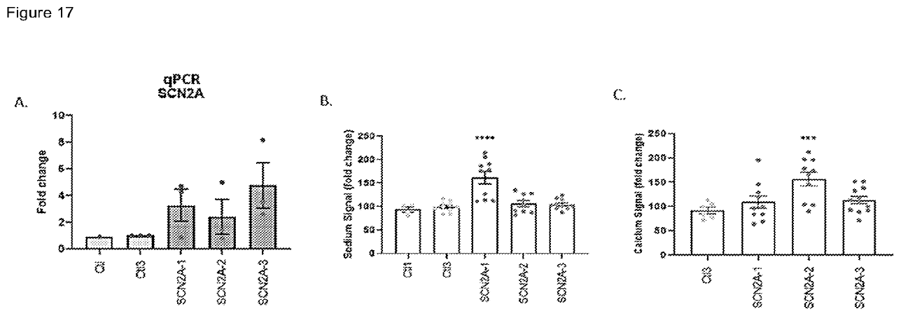

Expression of SCN2A in the iAstrocyte cells was measured by quantitative real time PCR (qRT-PCR). Unexpectedly, the three SCN2A patient lines, including the two cell lines predicted to be loss of function (LOF) mutations, had an increase trend in expression of SCN2A (FIG. 17A). As an indirect method of measuring the activity of the channel, the levels of sodium ions inside the cell was measured. The sodium ions were measured using a sodium indicator that emits a green signal when it binds to sodium ions. The integrated density of the fluorescent signal was quantified and normalized to the average of controls, and as can be observed in FIG. 17B, at least one of the LOF cell lines showed higher concentrations of sodium ions inside the cells, none of them shows reduced levels of sodium ions. This was unexpected and we are currently exploring several possible mechanisms including channel compensation (other sodium channels), as well as feedback loops that would affect expression levels of SCN2A.

As one of the main roles of Nav1.2 in iAstrocytes is to modulate calcium signaling, calcium signaling was measured in the iAstrocytes using a similar assay. At least one of the LOF cell lines demonstrated an increase in calcium signaling (FIG. 17C). These results are very intriguing as it suggests that some of the LOF mutations actually might cause a higher channel activity. This further suggests that patients can be further subgrouped based on channel activity within either a loss or gain of function mutation.

As abnormal sodium and calcium signaling can impact the overall stress level of the cell, families of SCN2A methods/read outs were previously reported and established to test therapeutic strategies for the in vitro studies involving iAstrocyte cells. These established methods/read outs involved measuring the levels of reactive oxidative species (ROS) within the iAstrocytes as well as mitochondria morphology and activity, as increase cellular sodium and calcium signaling can lead to alterations in mitochondria function. Automated image analysis was developed for these experiments by implementing computer vision algorithms. This will reduce human bias and help to increase the speed of testing moving forward.

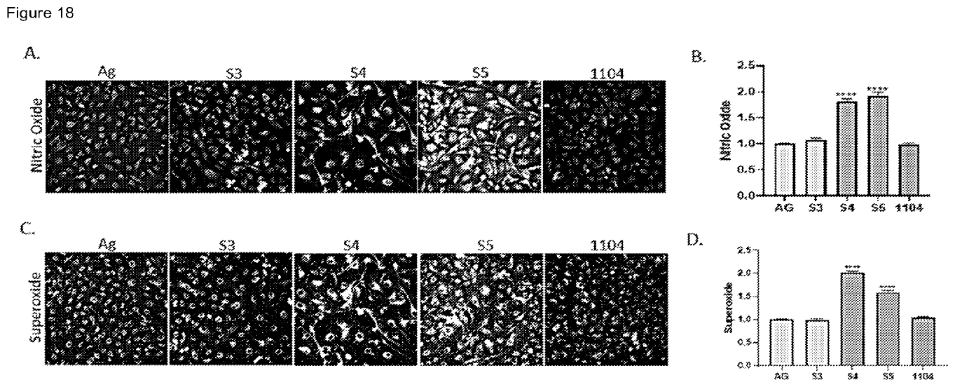

A fluorescent reactive oxidative species assay (ROS) was used to determine the amount of nitric oxide (NO) and superoxide being produced in SCN2A iAstrocytes cells to determine the stress level of the cell. As nitric oxide and superoxide production can be observed in FIGS. 18A and 18C, S4 (SCN2A-1, LOF) and S5 (SCN2A-3, LOF) cell lines have more nitric oxide and superoxide production in comparison to healthy controls (Ag adult control, and S3 child control). A computer vision algorithm was designed which averages fluorescent intensity readings as output for each field of view captured, both for cells and background as determined by the script; allowing us to accurately measure the true signal. Automated quantification of the signal intensity observed in the images demonstrate that the two LOF mutations cell lines have increased levels of NO and superoxide production, while the gain of function mutation did not show a change.

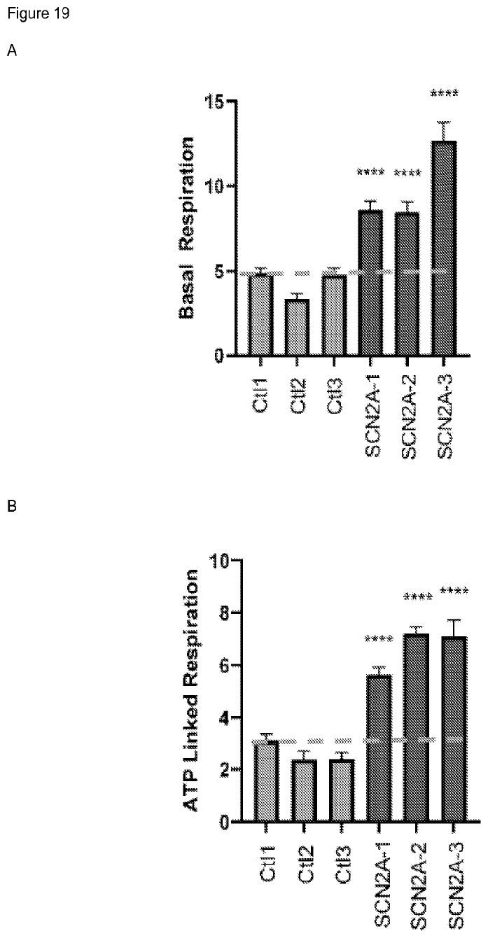

Preliminary observations made while characterizing SCN2A iAstrocytes suggest that mitochondrial dysfunction may play a role in the disease mechanism. Thus, the mitochondrial activity of iAstrocytes from SCN2A patients was evaluated. Induced astrocytes were seeded on seahorse plates to evaluate oxygen consumption following inhibition of the ATPase synthase, mitochondrial uncoupling and total shutdown of the electron transport chain, and oxygen consumption as described in Example 1. As shown in FIG. 19, these patients demonstrated an elevated basal respiration (FIG. 19A) and/or ATP-linked respiration (FIG. 19B). These findings suggest that SCN2A iAstrocytes have increased mitochondrial activity. Since metabolism is closely tied to these outcome measures, these findings also suggest that SCN2A iAstrocytes may have increased metabolic activity. The role of iAstrocytes in providing metabolic support for neurons and regulating neurotransmission suggests that these cells, in addition to neurons, may be a potential therapeutic target in patients with neurological disorders including SCN2A mutation-related disorders.

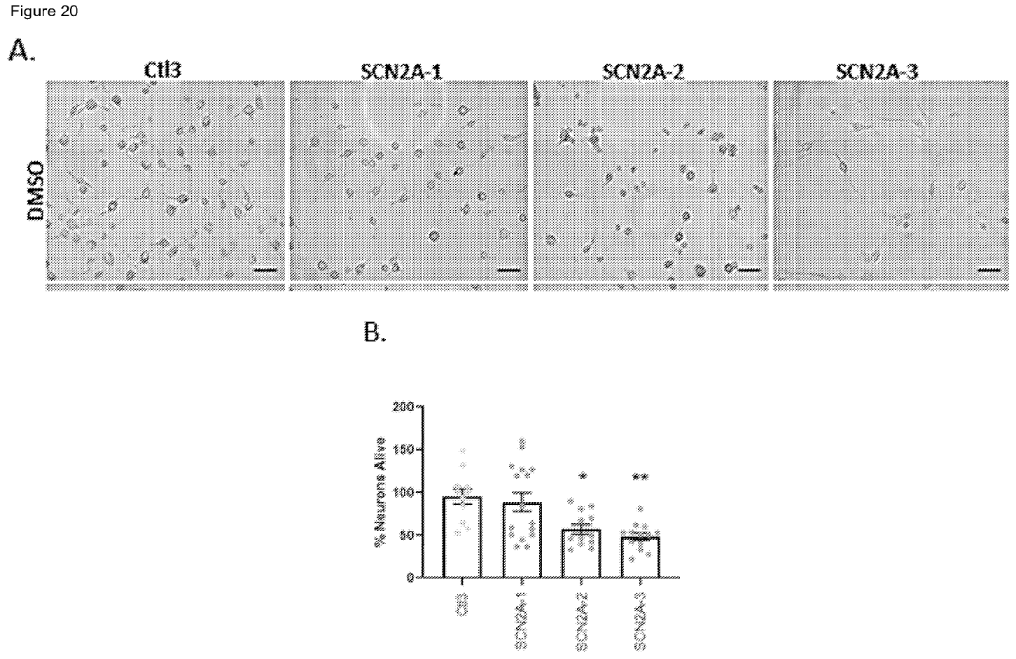

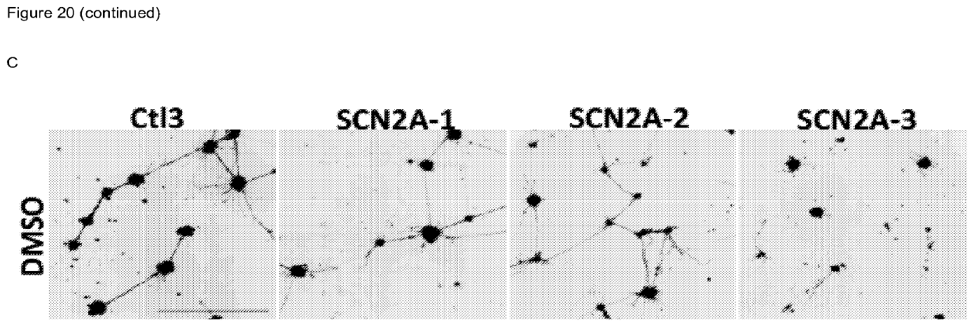

In addition to molecular characterization of the cell lines, it was also determined that the patient iAstrocytes were toxic to mouse neurons when cocultured together. FIG. 20C provides images of wild type GFP-neurons following 3 days in co-culture with patient SCN2A iAstrocyte cells as described in Example 1. Quantification of these neurons show a reduction in neuronal survival when cultured in the presence of SCN2A mutated iAstrocytes. This experiment demonstrated that SCN2A iAstrocytes show various degrees of toxicity to neurons (FIG. 20C).

### Example 7

iNeurons from Patients with SCN2A Mutations

Patient's fibroblasts were induced into induced neurons by small chemical compounds and in seven days we obtained immature neurons. Fibroblast cells were seeded on 12-well plates and cultured in human fibroblast medium (HFM). Fibroblast cells were then differentiated for 7 days into iNeurons in the presence of induction medium and treatment a chemical cocktail VCRFSGY (valproic acid; CHIR99021; Repsox; SP600125 (JNK inhibitor), G06983 (PKC inhibitor) and Y-27632 (ROCK inhibitor)). To verify differentiation, the iNeurons were immunostained for the neuronal marker, Tuj1. Representative bright field images of iNeurons following seven days of differentiation are shown in FIG. 20A. There was reduce neuron survival in two SCN2A patient lines (SCN2A-2 and SCN2A-3) following differentiation (FIG. 20A).

The induced neurons expressed neuronal markers such as Tuj1, Map2, GABA and Syn1. These cells represent a mixed neuronal population of different subtypes. The data suggests neuronal health of SCN2A iNeurons was impacted in some of the patient cell lines (FIGS. 20A and 20B) as less neurons were alive by day seven when compared to control. Quantification of neuron survival percentage was determined by the amount of neuronal marker Tuj1 positive cells (percent of initial cells) remaining at day seven compared to control. Three to five images were taken per condition and counting was performed by hand by a blinded experimenter.

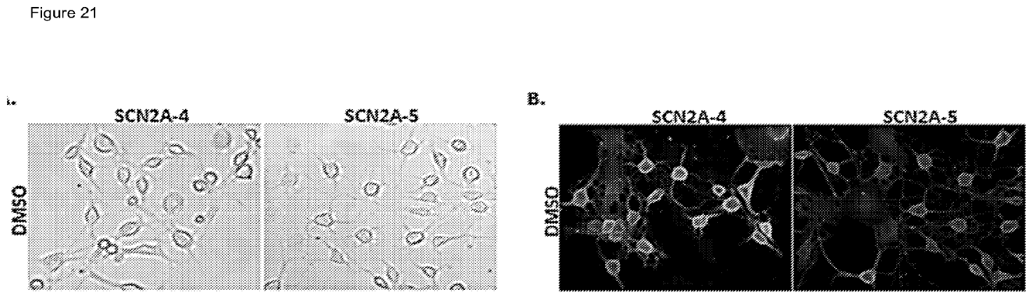

In addition to SCN2A-1 (S4), SCN2A-2 (S5) and SCN2A-3 (1104), two additional cell lines: SCN2A-4 (038) and SCN2A-5 (048) were converted to iNeurons. These fibroblast cells were also directly converted to iNeurons using small chemical compounds. Interestingly, we observed that these patient induced neurons have distinct neuronal characteristics such as different soma size, branching and neurite lengths (FIG. 21A). Similar to previous observations, an increased expression of Nav1.2 was observed in SCN2A-4 iNeurons (FIG. 21B) when compared to SCN2A. Subtle differences at dendritic branch points can drastically alter the ability of synaptic input to generate, propagate, and time action potentials.

In general, SCN2A-4 and SCN2A-5 iNeurons have more branching and neurites than observed for the other SCN2A lines SCN2A-1, SCN2A-2 and SCN2A-3. SCN2A-4 and SCN2A-5 last longer than SCN2A-2 and SCN2A-3 iNeurons.

### Example 8

Human Brain Organoids

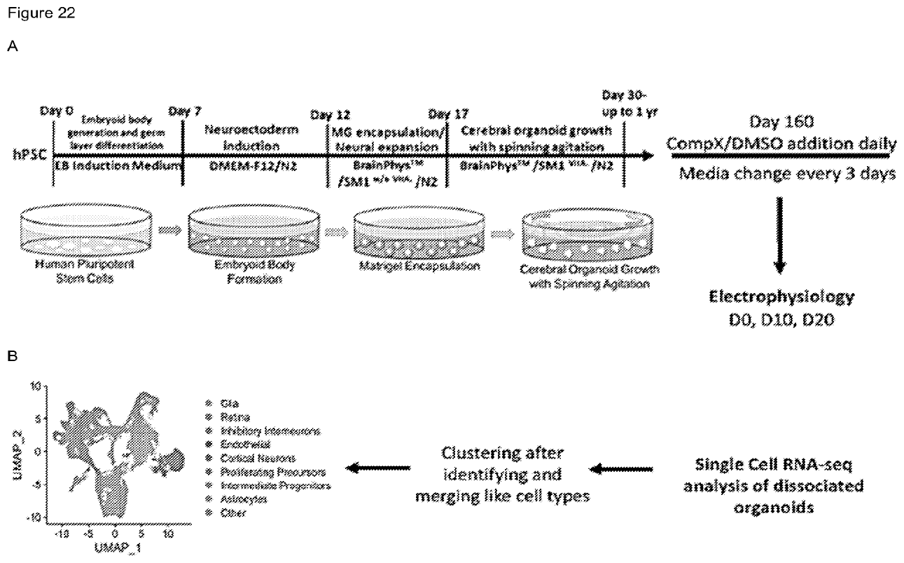

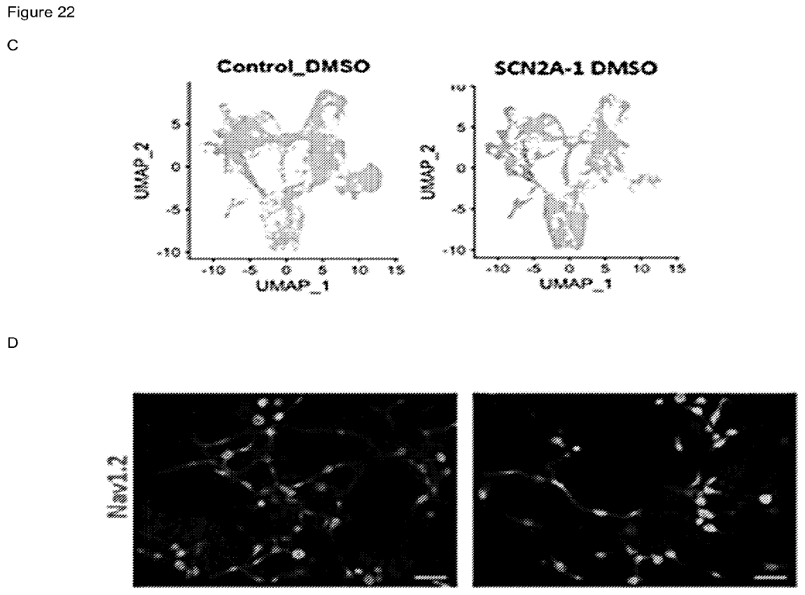

As 3D organoids provide the environment that neurons need to form dynamic neural networks, as well as neuronal diversity more similar to a developing brain, additional valuable information can be gained using this model system. SCN2A human pluripotent stem cells (hPSC) cells were converted into brain organoids as shown FIG. 22 and as follows. hPSC were induced to form embryoid bodies (EB) by use of EB induction media (day 0-6). Neuroectoderm induction was further promoted by switching from EB induction media to neural induction media (day 7-11). Media was switched to differentiation media and EBs were encapsulated in matrigel droplets to promote neural expansion (day 12-16). Differentiation media was continued to promote cerebral organoid growth with spinning agitation (day 17-30) as described in FIG. 22A. Induced pluripotent stem cells were generated from patient within mutation in SCN2A-1 and used to generate brain organoids. The reprogramming method used in this study differed from the reprogramming method used in the studies described in Example 1. The brain organoids were grown for 120 days.

At day 160, the organoids were dissociated, and single cell RNA sequencing was carried out. Electrophysiology readings were taken on day 0, day 10, and day 20 during the treatment period. Following the 20^{th} day of treatment, organoids were dissociated and single cell RNA-sequence analysis was performed using the 10× genomics platform. Cell clusters were defined using a hierarchical approach, first identifying neuronal vs. non-neuronal populations, then narrowing the definition based on specific markers, and finally merging clusters representing the same broad cell types. Clusters of distinct cell types were mapped as shown in FIG. 22B. Uniform manifold approximation and projection (UMAP) for dimension reduction was used for visualization.

The cells were then sorted and clustered according to their expression profiles with merged clusters representing the same broad cell types (see FIG. 22B). Similar to the data provided in the iAstrocyte model in the organoid model a higher expression of SCN2A in the patient cell line compared to healthy controls was observed (FIG. 22C). Thus, this a different model system confirms that this LOF mutation leads to increased SCN2A expression levels using cells that were generated with a different reprogramming method. Consistent with other reports, SCN2A expression was higher in cortical and inhibitory interneuron cluster group. This expression pattern was also observed by immunofluorescence staining in the SCN2A-1 induced neurons model.

### Example 9

Characterization iAstrocytes from CLN3 Patients

The cellular mechanisms by which mutations in a CLN gene causes Batten disease is not well understood. In particular, the cellular mechanism by which mutations in the CLN3 gene are poorly understood and focused on the function and expression of CLN3 in neurons, with little known about the contributions of other cell types to disease mechanisms. In vitro modelling escribed herein allows for investigation of the physiological pathways, and cellular interactions involved in the progression and mechanism of Batten disease.

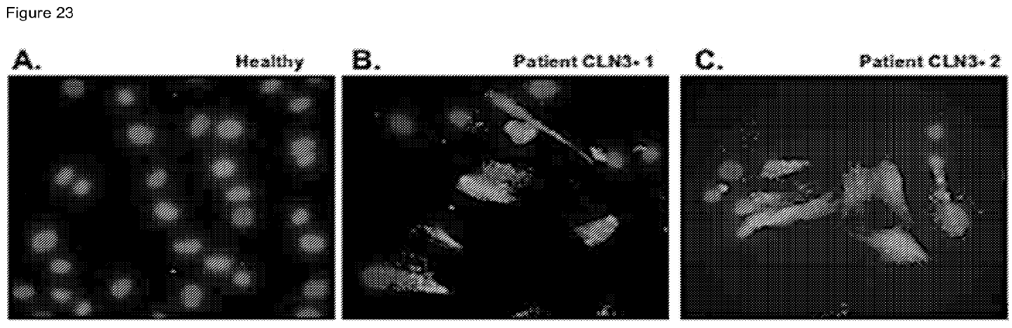

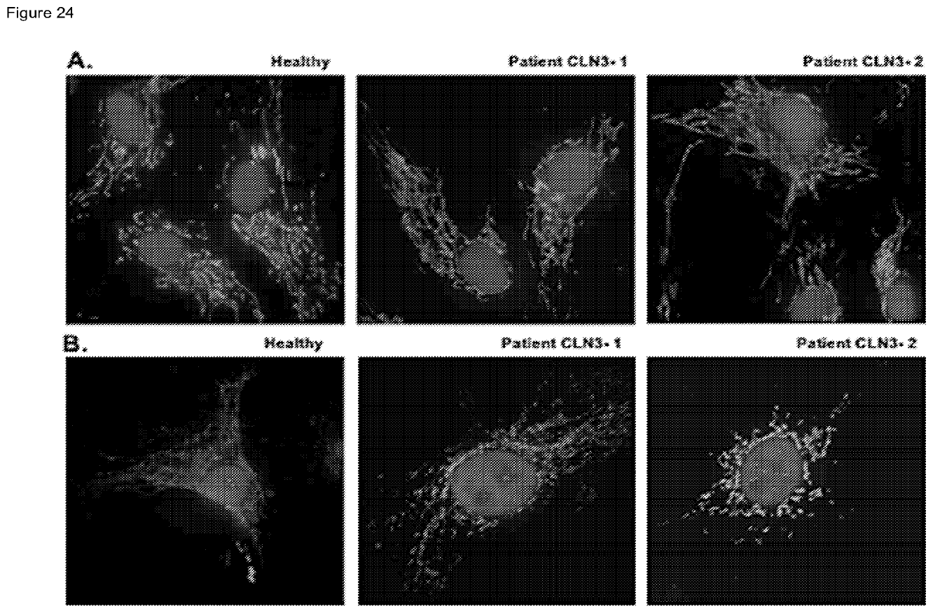

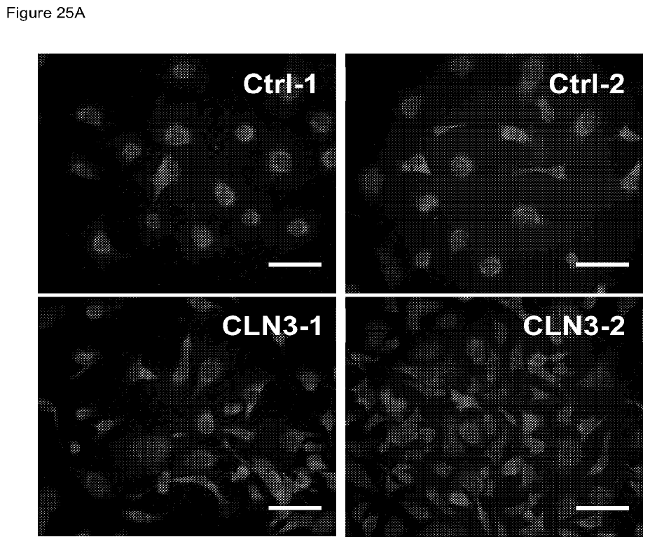

Fibroblasts were obtained from 2 CLN3 patients. As shown in FIG. 23, these fibroblasts demonstrated autofluorescent storage material accumulation. The fibroblasts were converted to induced neuronal progenitor cells (iNPCs), and these neuronal progenitor cells were differentiated to iAstrocytes as described in Example 1. The cell lines are referred to as CLN3-1 and CLN3-2.

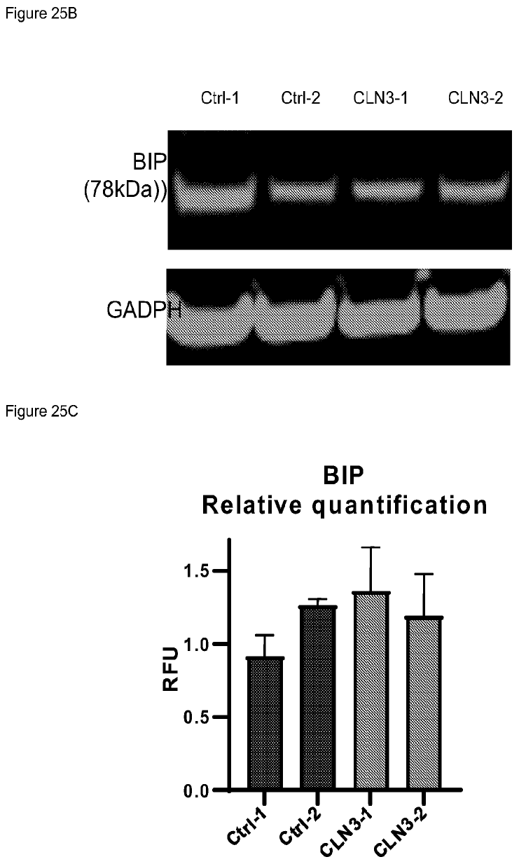

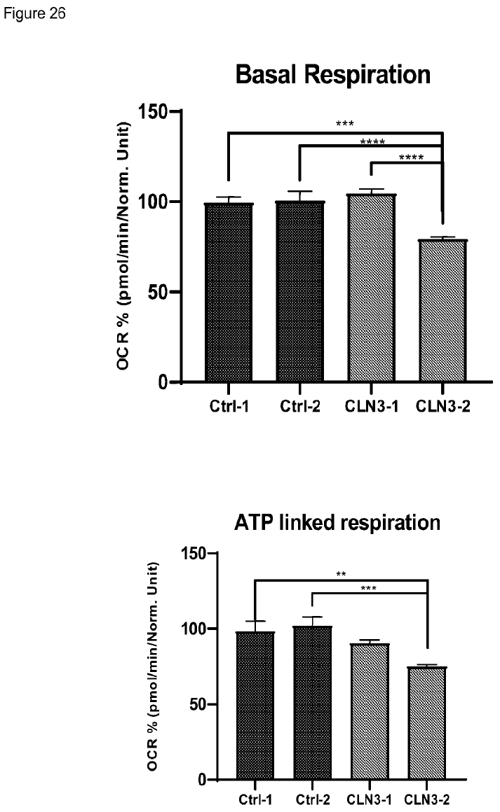

The mitochondrial morphology of the iAstrocytes was investigated using immunohistochemistry. iAstrocytes were seeded on glass cover slips or plastic chamber slides and immunostained for complex IV (CoxIV). Immunostains were imaged using a Nikon microscope. As shown in FIG. 24, the CLN3 iAstrocytes have abnormal mitochondrial morphology in which the mitochondrial network is fragmented and rounded, and this abnormal mitochondrial morphology was specific to the iAstrocytes. However, there was no difference in ER stress marker BIP (FIG. 25).

Mitochondrial activity of the CLN3 patient iAstrocytes was measured as basal respiration and ATP linked respiration. Oxygen consumption rate (OCR) was measured using the Seahorse XF and Seahorse XFe for extracellular flux analysis as described in Example 1 As shown in FIG. 26, the mitochondrial morphology changes observed in the iAstrocytes were indicative of changes in mitochondrial.

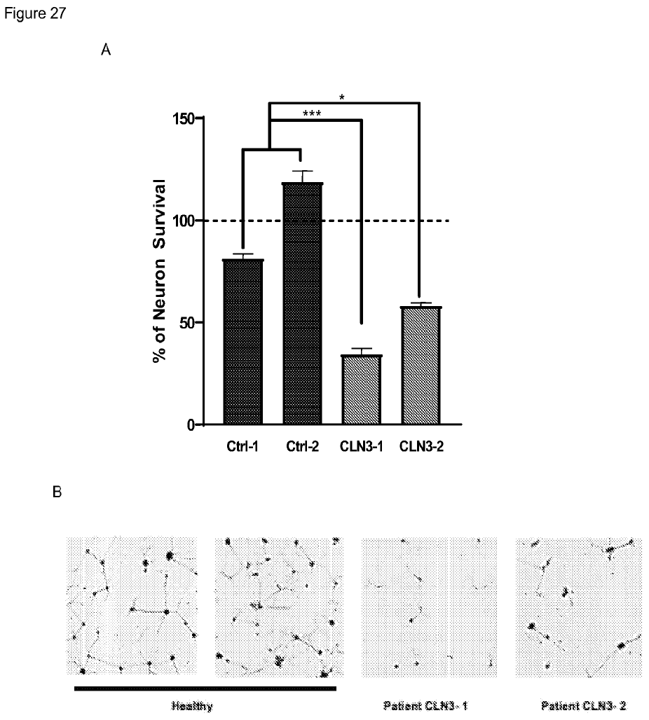

Co-culture assays with healthy neurons were carried out as described in Example 1. Healthy mouse GFP+ neurons were plated with CLN3 patient derived iAstrocytes and healthy controls. FIG. 27A, the percentage of neuron surviving after 3 days of co-culture was reduced by the iAstrocytes from CLN3 patients. Thus, the CLN3 derived iAstrocytes were were toxic to neurons, with some lines showing a more toxic phenotype than others. FIG. 26B provides representative images of the GFP+ neurons 3 days after the culture.

### Example 10

Characterization of iAstrocytes from NEDAMSS Patients

Fibroblasts from patients suffering from NEDAMSS (neurodevelopmental disorder with regression, abnormal movements, loss of speech, and seizures) were converted to induced neuronal progenitor cells (iNPCs) as previously described (Meyer et al., PNAS 829-832 (2014)). The fibroblasts were obtained from 4 families having nonsense variants in the IRF2BPL gene resulting in the truncation of its RING finger domain. The IRF2BPL gene mutations represented are summarized below.

| | | Unaffected |
|---|---|---|
| Family | Mutation | family members |
| 1 | Proband with E172X and G195V | Unaffected parents |
| 2 | Proband with Y173X variant | Three unaffected |
| | | family members |
| | | (parents + sib) |
| 3 | Proband with R188X variant | Unaffected parents |
| 4 | Proband with A708fs variant | Three unaffected |
| | | family members |
| | | (parent + sib) |

| Cell Line | Description |
|---|---|
| H1 | Healthy unrelated boy (S3) |
| H2 | Healthy sister (542) |
| H3 | Healthy adult female (AG) |
| H4 | Healthy adult male (fTM154) |
| P1 | Child patient with stop codon mutation near |
| | N terminal (537) |
| P2 | Child patient with stop codon mutation near |
| | N terminal (152) |
| P3 | Adult patient with stop codon mutation near |
| | N terminal (1911) |
| P4 | Child patient with frameshift mutation near |
| | C terminal (645) |

Neuronal progenitors' cells were cultured on fibronectin coated dishes in NPC media (DMEM/F12 media containing 1% N2 supplement (Life Technologies), 1% B27, 1% Anti-anti (antibiotic-antimycotic) 20 ng/ml fibroblast growth factor-2) until confluent. iAstrocytes were differentiated by seeding a small quantity of NPCs on another fibronectin coated dish in iAstrocyte inducing media (DMEM media containing 0.2% N2). These induced astrocytes are referred to as iAstrocytes or iAST herein. Five days post differentiation, induced astrocytes were seeded either into a 96 well (10,000 cells/well), 384 well (2,500 cells/well), a 24 well seahorse plate (20,000 cells/well) or a 96 well seahorse plate (10,000 cells/well).

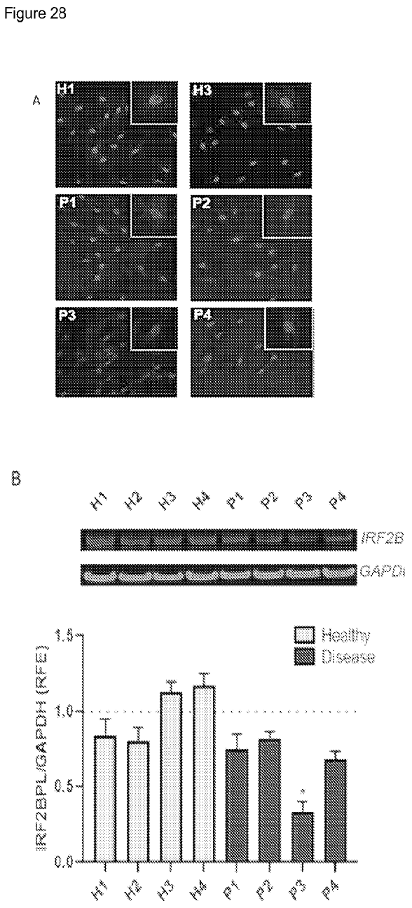

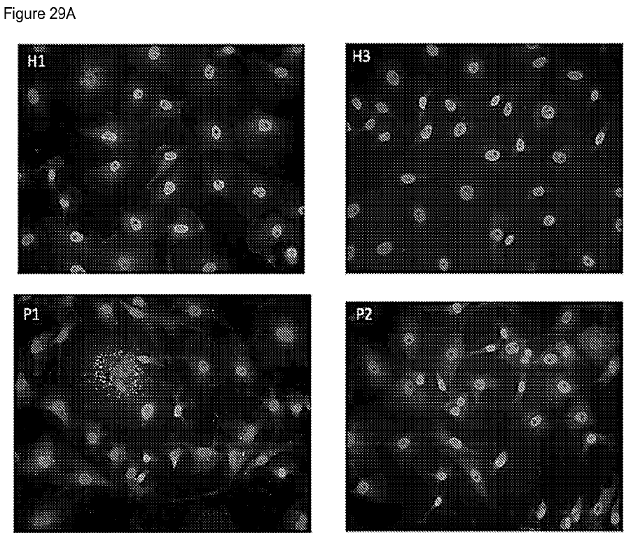

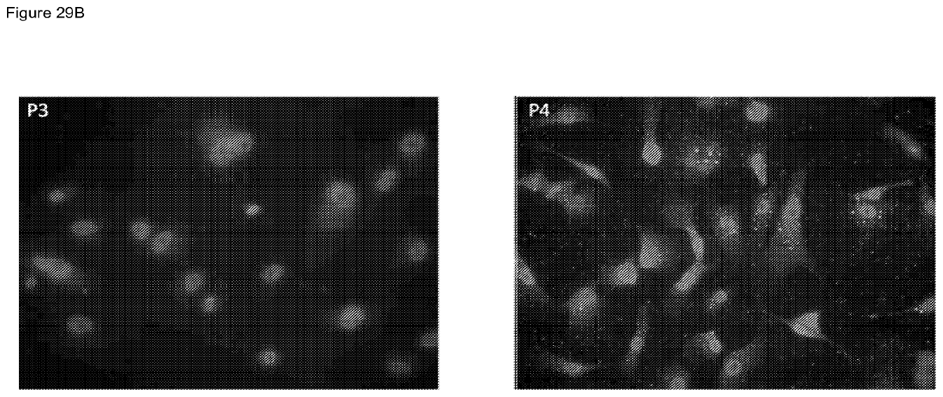

Immunohistochemistry and western blot analysis was carried out on the primary fibroblasts from NEDAMSS patients using an antibody specific for IRF2BPL (Novus Biologics). As shown in FIG. 28, expression of the IRF2BPL protein was not significantly different in the primary fibroblasts from the NEDAMSS patients (P1,P2 and P4) except the cells from patient P3 which showed reduced expression, compared to fibroblasts from healthy individuals (H1,H2,H3 and H4).

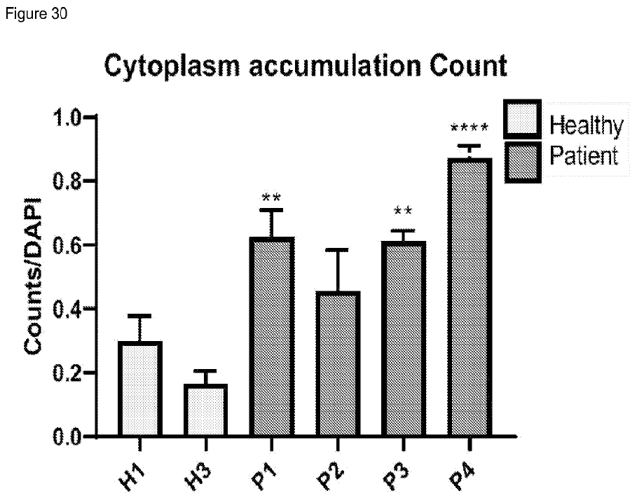

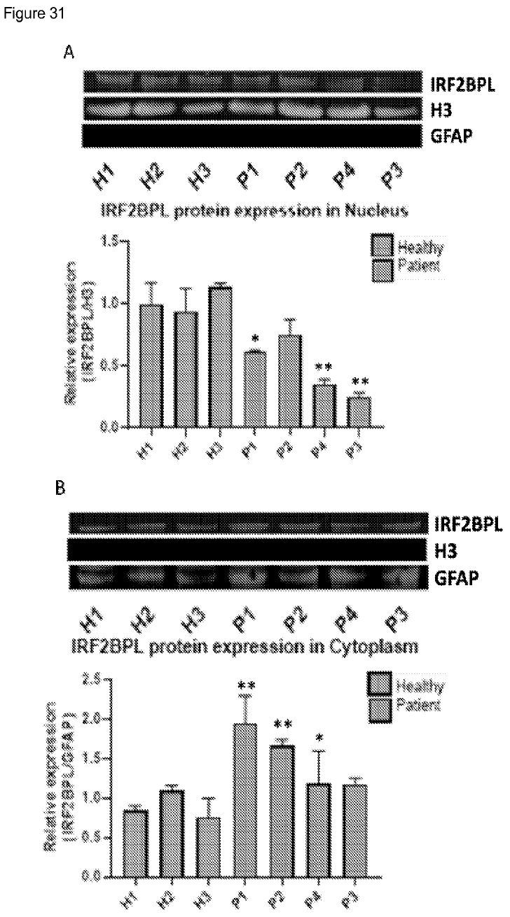

FIGS. 29A and 29B provide representative photos of the immunohistochemistry staining for IRF2BPL and DAPI for the cell nucleus, and FIG. 30 provides quantification of the aberrant cytoplasmic accumulation as observed from the immunofluorescence images. The normalized ratio is the number of cells with cytoplasm accumulation of IRF2BPL in iAstrocytes to the DAPI counts (n=3). Blinded-hand counting was carried out by two independent researchers. There was a clear difference in cytoplasmic IRF2BPL protein localized in patient cell lines. The photos and the graphs provided in FIG. 31 demonstrate that the IRF2BPL accumulated more in the cytoplasm of the iAstrocytes derived from the NEDAMSS patients (P1, P2, P3 and P4) rather than localizing mostly to the nucleus of the iAstrocytes like the healthy individuals (H1 and H3). NPER extraction kit was used to separate the two extracts and confirmed accumulation of the protein in the cytoplasm in patient iAstrocytes.

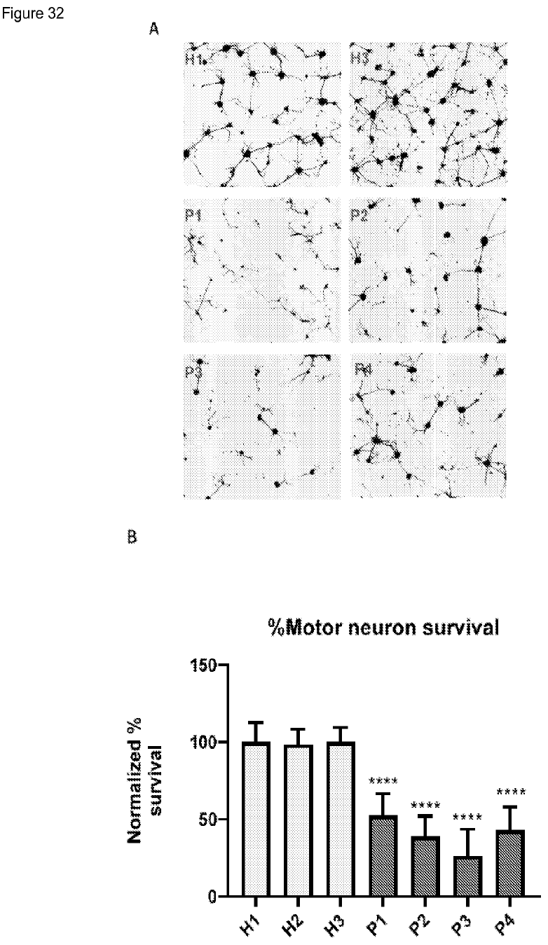

Coculture of iAstrocytes with mouse stem cell derived GFP positive motor neurons (according to publication Meyer et al, PNAS 2014). Briefly, iAstrocytes were plated in a 96 well plate to form a monolayer. The next day 10K, FACS sorted gfp positive mouse motor neurons are added to each well. Survival and morphology of neurons are monitored using the INCELL6000 automatic imager and analyzer software for 3 days. It was determined that iAstrocytes from NEDAMSS patients were toxic or less supportive to the motor neurons compared to iAstrocytes from healthy controls. FIG. 32A provides representative photos of the motor neurons in the coculture with iAstrocytes from NEDAMSS patients and healthy individuals and FIG. 32B shows the percentage motor neuron survival. NEDAMSS iAstrocytes show significantly reduced motor neuron survival in the cocultures compared to healthy iAstrocytes on day 3. Of note, only the motor neurons are visible as they contain GFP (represented in black). In these photos, the motor neurons are visible due to GFP expression and the iAstrocytes are not visible.

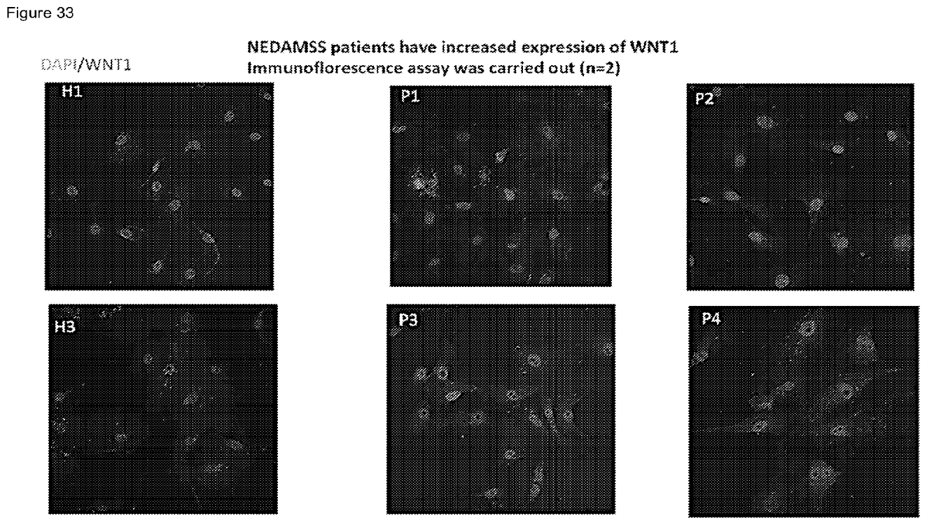

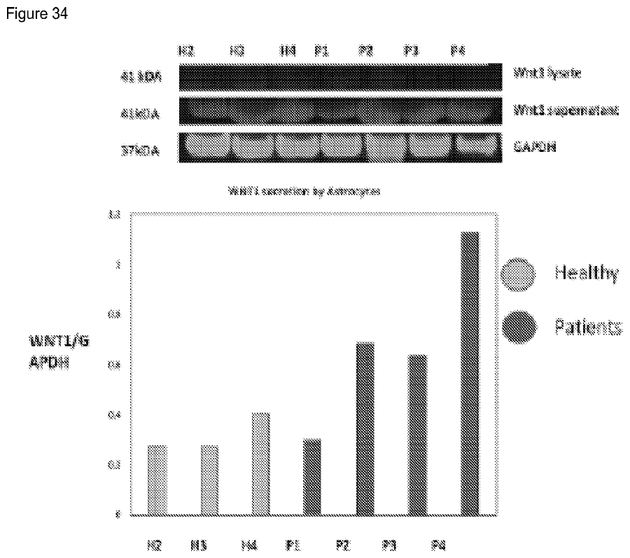

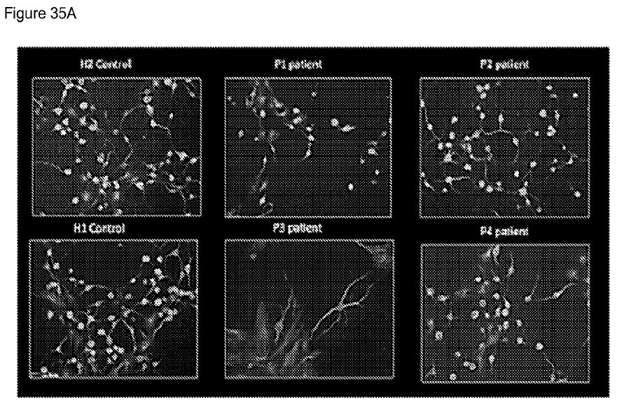

FIGS. 33 and 34 demonstrate that the NEDAMSS patients have increased secretion of WNT1 compared to healthy patients. Dysregulation of wnt signaling pathway could lead to neurodegeneration in NEDAMSS patients.

### Example 11

Neurons from NEDAMSS Patients have Reduced Survival

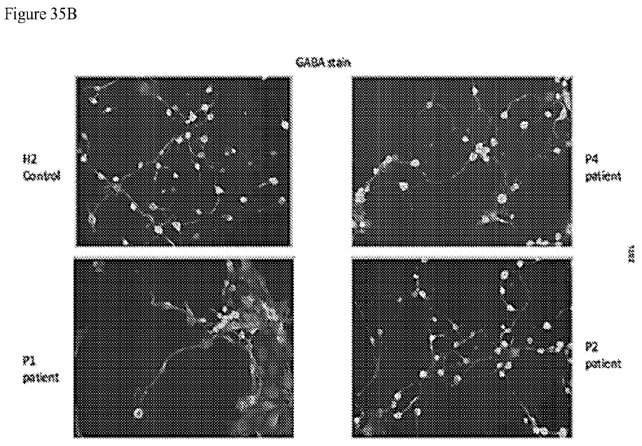

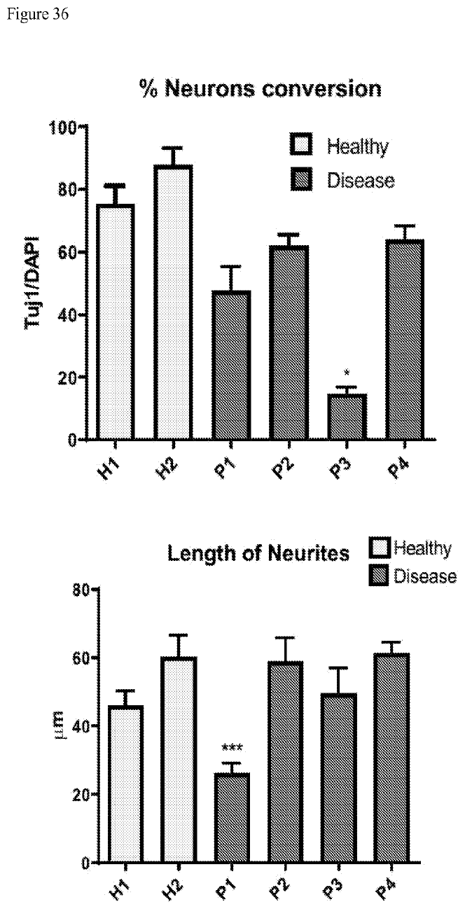

Fibroblasts isolated from healthy individuals and NEDAMSS patients were differentiated to neurons as described in Hu et al., Cell Stem Cell, 17(2):204-12., 2015, the disclosure of which is incorporated herein by reference in its entirety. The fibroblasts were incubated with 7 small molecules as described in Hu et al (supra) for 7 days. This method does not use transcriptional factor-expressing virus. FIGS. 35A and 35B provide representative photos showing that the neurons induced from fibroblasts from NEDAMSS patients had reduced survival or reduced differentiation capacity. FIG. 35A shows staining for the pan-neuron marker Tuj1 from NEDAMSS patients (P1, P2, P3 and P4) and healthy individuals (H1 and H2). FIG. 35B show staining for the neuronal subtype marker Gaba in neurons from NEDAMSS patients (P1, P2 and P3) and a healthy individual (H2). FIG. 36 provides quantification of the percent number of Tuj1+ neurons (Tuj1) normalized to DAPI and the length of the neurites from the NEDAMSS patients (P1, P2, P3 and P4) and healthy individuals (H1 and H2) on day 7 of differentiation.

### Example 12

Characterization of iAstroctyes from Pitt Hopkins Patients

Direct conversion of patient fibroblasts to neuronal progenitor cells (NPCs) allows for the study of disease mechanism in specific cell types of interest. This in vitro cell model can be used to distinguish patient responders based on the presence of specific disease markers of cellular stress. If disease markers are present, this information can then be used to choose potential therapeutics from a selection of therapeutic molecules, such as small molecules or biologics to determine their effect on the Pitt Hopkins Syndrome (PTHS) phenotype.

Fibroblasts from six PTHS patients containing either heterozygous missense or deletion mutations in TCF4 were obtained and are summarized in Table 4 below. The fibroblasts were converted to induced neuronal progenitor cells (iNPCs) using retroviruses, SOX2, KLF4, cMyc, and Oct3/4, and chemically defined media as previously described (Meyer et al., PNAS 829-832 (2014)). Subsequently, the NPCs were differentiation into iAstrocytes (iAstrocytes). Neuronal progenitor cells were cultured on fibronectin coated dishes in NPC media (DMEM/F12 media containing 1% N2 supplement (Life Technologies), 1% B27, 1% Anti-anti (antibiotic-antimycotic) 20 ng/ml fibroblast growth factor-2) until confluent. iAstrocytes were differentiated by seeding a small quantity of NPCs on another fibronectin coated dish in iAstrocyte inducing media (DMEM media containing 0.2% N2). These induced iAstrocytes are referred to as iAstrocytes or iAST herein. Neurons were converted from NPCs by transduction with retro-Ngn2.

TABLE 4

| Cell Line | Sex | Mutation |
|---|---|---|
| TCF4-1 | female | c.1486 + 5 g > T |
| TCF4-2 | male | c.520C > T(p.Arg174X) |
| TCF4-3 | male | Heterozygous gene deletion |
| TCF4-4 | female | c.1726C > T (p.Arg576X) |
| TCF4-5 | male | C.624delc |
| TCF4-6 | male | c.550-2A > G |

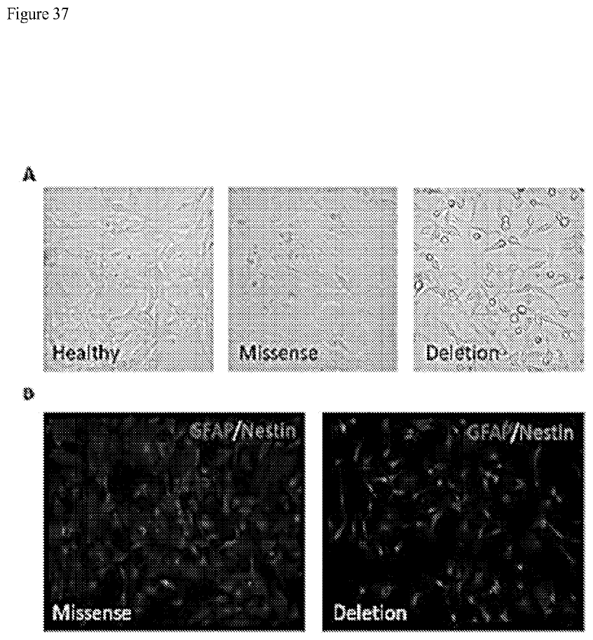

Five days post differentiation, induced astrocytes were seeded either into a 96 well (10,000 cells/well), 384 well (2,500 cells/well), a 24 well seahorse plate (20,000 cells/well) or a 96 well seahorse plate (10,000 cells/well) as described in Example 1. A representative image of iAstrocytes from healthy and TCF4 mutants following differentiation are provided in FIG. 37. Interestingly, PTHS iAs with mutations leading to a gene deletion also had a negative impact on iAstrocyte differentiation. The impact of TCF4 deletion mutation on astrocyte differentiation was further assessed by immunostaining. Here TCF4 deletion show reduced astrocyte marker, GFAP, and elevated levels of neuronal progenitor marker, Nestin when compared to missense mutations.

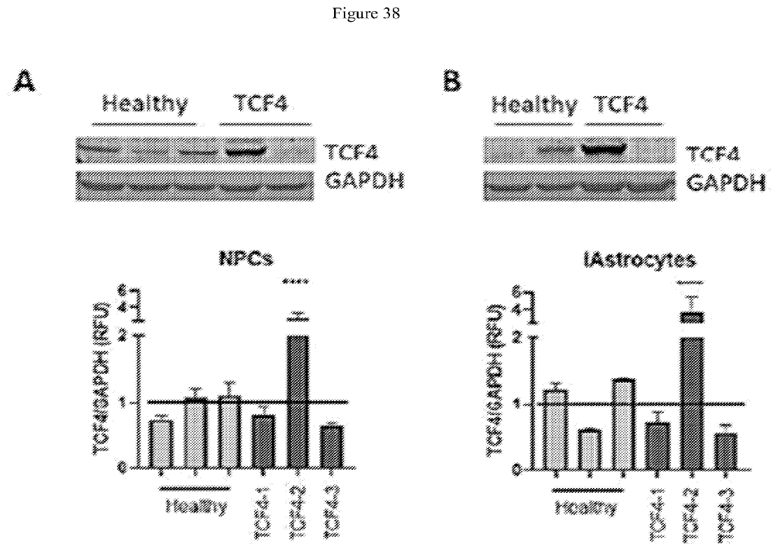

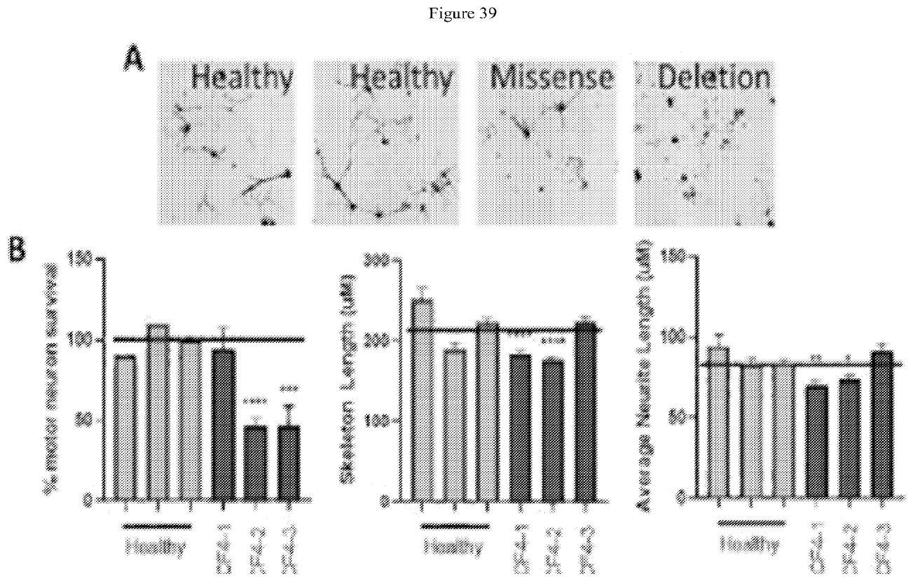

Initial studies on three of these patient lines investigated the levels of TCF4 protein in patient neuronal progenitor cells and iAstrocytes. Western blot of TCF4 (isoforms B,D,E,F,M,N,O,Q) discovered differential levels in PTHS iAstrocytes and NPCs compared to healthy controls (FIGS. 38A and B). Importantly, patients with heterozygous genetic deletions had 50% reduction in TCF4 levels whereas missense mutations either lead to no change in protein levels or significant upregulation, potentially suggesting toxic overexpression (FIG. 39B).

In addition, GFP+ neurons co-cultured with iAstrocytes from TCF4 patients show reduced neuronal survival (FIGS. 39A and 13,1.). PTHS iAstrocytes caused changes in neuronal morphology (FIG. 39B). Thus, this direct conversion technology and co-culture assay can be utilized to identify new disease mechanisms as well as evaluate potential therapeutic strategies (including but not limited to gene therapy) to treat patients with PTHS.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed:

1. A method of predicting progression of a neurological or neurodegenerative disease in a subject in need comprising detecting a modification of one or more disease markers in a glial cell or neuronal cell generated from a skin cell of the subject, compared to a glial cell or neuronal generated from a control cell.

2. A method of identifying a subject who is responsive to a therapeutic agent, comprising detecting a modification of one or more disease markers in a glial cell or neuronal cell generated from a skin cell of the subject, compared to a glial cell or neuronal cell generated from a control cell and wherein the modification is indicative of cells that are responsive to the therapeutic agent.

3. A method of determining effectiveness of a therapeutic agent in a subject, comprising detecting a modification of one or more disease markers in a glial cell or neuronal cell generated from a skin cell of the subject obtained from the subject after administration of the therapeutic agent, compared to a modification of one or more disease markers in a glial cell or neuronal cell generated from a skin cell of the subject obtained from the subject before administration of the therapeutic agent.

4. The method of any one of claims 1-3, wherein the glial cell or neuronal is differentiated from a neuron progenitor cell derived from a skin cell obtained from the subject.

5. The method of any one of claims 1-4 wherein the glial cell is an astrocyte, microglia or oligodendrocyte.

6. The method of claim any one of claims 1-4 wherein the skin cell is a fibroblast.

7. The method of any one of claims 1-6 wherein the modification is an increase in one or more disease markers, and wherein the increase is indicative of an increased progression.

8. The method of any one of claims 1-6 wherein the modification is a decrease in one or more disease markers disease markers, wherein the decrease is indicative of an increased progression.

9. The method of any one of claims 1-8 wherein the disease marker is a marker for Alzheimer's disease, Parkinson's disease, Multiple Sclerosis, Amyotrophic Lateral Sclerosis (ALS), other demyelination related disorders, senile dementia, subcortical dementia, arteriosclerotic dementia, AIDS-associated dementia, or other dementias, a central nervous system cancer, traumatic brain injury, spinal cord injury, stroke or cerebral ischemia, cerebral vasculitis, epilepsy, Huntington's disease, Rett Syndrome, Pitt Hopkins Syndrome, SMARD1/CMT25, Tourette's syndrome, Guillain Barre syndrome, Wilson disease, Pick's disease, neuroinflammatory disorders, SCN2A-related disorders, encephalitis, encephalomyelitis or meningitis of viral, fungal or bacterial origin, or other central nervous system infections, prion diseases, cerebellar ataxias, cerebellar degeneration, spinocerebellar degeneration syndromes, Friedreichs ataxia, ataxia telangiectasia, spinal dysmyotrophy, spinal muscle atrophy, NEDAMSS, SCN2A-related disorders, SLC6A1-related disorders, SCN1A-related disorder, IRF2BPL-related disorders, progressive supranuclear palsy, dystonia, muscle spasticity, tremor, retinitis pigmentosa, striatonigral degeneration, mitochondrial encephalo-myopathies, neuronal ceroid lipofuscinosis, Batten Disease, hepatic encephalopathies, renal encephalopathies, metabolic encephalopathies, toxin-induced encephalopathies, or radiation-induced brain damage,

10. The method of any one of claims 1-9 wherein the disease marker is abnormal mitochondria morphology, change in basal respiration, change in ATP linked respiration, glutamate, or in vitro motor neuron toxicity.

11. The method of any one of claims 1-8 wherein the disease marker is a marker of ALS, and the marker is p62, SOD1, misfolded SOD1, BIP, TDP43, abnormal mitochondria morphology, change in basal respiration, change in ATP linked respiration, mi-RNA-146, glutamate, or in vitro motor neuron toxicity.

12. The method of any one of claims 1-8 wherein the disease marker is a marker of Batten Disease and the marker is accumulation of auto fluorescent storage material, abnormal mitochondria morphology, change in basal respiration, change in ATP linked respiration, or in vitro motor neuron toxicity.

13. The method of any one of claims 1-8 wherein the disease marker is a marker of a SCN2A-related disorder and the marker is nitric oxide or superoxide, abnormal mitochondria morphology, change in basal respiration, change in ATP linked respiration, or in vitro motor neuron toxicity.

14. The method of any one of claims 1-8 wherein the disease marker is a marker of NEDAMSS and the marker is wnt1, IRF2BPL, abnormal mitochondria morphology, change in basal respiration, change in ATP linked respiration, or in vitro motor neuron toxicity.

15. The method of any one of claims 1-8 wherein the disease marker is a marker of SLC6A1-related disorder and the marker abnormal mitochondria morphology, change in basal respiration, change in ATP linked respiration, or in vitro motor neuron toxicity.

16. The method of any one of claims 1-8 wherein the disease marker is a marker of SCN1A-related disorder and the marker abnormal mitochondria morphology, change in basal respiration, change in ATP linked respiration, or in vitro motor neuron toxicity.

17. The method of any one of claims 1-8 wherein the disease marker is a marker of Pitt Hopkins Syndrome and is a marker TCF-4 or in vitro motor neuron toxicity.

18. The method of any one of claims 1-17 further comprising the step of obtaining skin cells from the subject.

19. The method of any one of claims 1-18 further comprises the step of generating induced neuronal progenitor cells (iNPCs) from skin cells obtained from the subject.

20. The method of claim 20 further comprising the step of differentiating iNPCs to Astrocytes and/or neurons and/or oligodendrocytes

21. The method of any one of claims 1-20 wherein the subject is suffering from, is at risk of suffering from or has been diagnosed with Alzheimer's disease, Parkinson's disease, Multiple Sclerosis, Amyotrophic Lateral Sclerosis (ALS), other demyelination related disorders, senile dementia, subcortical dementia, arteriosclerotic dementia, AIDS-associated dementia, or other dementias, a central nervous system cancer, traumatic brain injury, spinal cord injury, stroke or cerebral ischemia, cerebral vasculitis, epilepsy, Huntington's disease, Rett Syndrome, Pitt Hopkins Syndrome, SMARD1/CMT25, Tourette's syndrome, Guillain Barre syndrome, Wilson disease, Pick's disease, neuroinflammatory disorders, SCN2A-related disorders, encephalitis, encephalomyelitis or meningitis of viral, fungal or bacterial origin, or other central nervous system infections, prion diseases, cerebellar ataxias, cerebellar degeneration, spinocerebellar degeneration syndromes, Friedreichs ataxia, ataxia telangiectasia, spinal dysmyotrophy, spinal muscle atrophy, NEDAMSS, progressive supranuclear palsy, dystonia, muscle spasticity, tremor, retinitis pigmentosa, striatonigral degeneration, mitochondrial encephalo-myopathies, neuronal ceroid lipofuscinosis such as Batten Disease, hepatic encephalopathies, renal encephalopathies, metabolic encephalopathies, toxin-induced encephalopathies, or radiation-induced brain damage.

22. The method of any one of claims 1-21 wherein the subject has been diagnosed with ALS or has a mutation in SOD1, TDP43, C90RF72 or FUS.

23. The method of any one of any one of claims 1-21 wherein the subject has been diagnosed with Batten Disease or neuronal ceroid lipofuscinosis, or has a mutation in CLN1 gene, CLN2 gene, CNL3 gene, CLN4 gene, CLN5 gene, CLN6 gene, CLN7, CLN8 or CLN10.

24. The method of any one of claims 1-21 wherein the subject has SCN2A mutation, a mutated SCN2A voltage-gated sodium channel protein a SLC6A1 mutation, a SCN1A mutation or a mutation in IRF2BPL.
